SEQUENCE LISTING (33 sequences)

SEQ ID NO: 1 - protein, 183 aa, synthetic construct
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 2 - protein, 133 aa, synthetic construct
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3 - RNA, 552 nt, synthetic construct
  source 1..552: mol_type = other RNA
atggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 4 - RNA, 402 nt, synthetic construct
  source 1..402: mol_type = other RNA
atggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 5 - protein, 253 aa, synthetic construct
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 239 aa, synthetic construct
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - protein, 215 aa, synthetic construct
MWPPGSASQPPPSPAAATGLHPAARPVSLQCRLSMCPARSLLLVATLVLLDHLSLARNLPVATPDPGMFPCLHHSQNLLRAVSNMLQKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - protein, 219 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 9 - RNA, 762 nt, synthetic construct
  source 1..762: mol_type = other RNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 10 - RNA, 720 nt, synthetic construct
  source 1..720: mol_type = other RNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 11 - RNA, 648 nt, synthetic construct
  source 1..648: mol_type = other RNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 12 - RNA, 660 nt, synthetic construct
  source 1..660: mol_type = other RNA
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 13 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 14 - RNA, 986 nt, synthetic construct
  source 1..986: mol_type = other RNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtta

SEQ ID NO: 15 - protein, 208 aa, synthetic construct
MDPFLVLLHSVSSSLSSSELTELKFLCLGRVGKRKLERVQSGLDLFSMLLEQNDLEPGHTELLRELLASLRRHDLLRRVDDFEAGAAAGAAPGEEDLCAAFNVICDNVGKDWRRLARQLKVSDTKIDSIEDRYPRNLTERVRESLRIWKNTEKENATVAHLVGALRSCQMNLVADLVQEVQQARDLQNRSGAMSPMSWNSDASTSEAS

SEQ ID NO: 16 - RNA, 627 nt, synthetic construct
  source 1..627: mol_type = other RNA
atggacccgttcctggtgctgctgcactcggtgtcgtccagcctgtcgagcagcgagctgaccgagctcaagttcctatgcctcgggcgcgtgggcaagcgcaagctggagcgcgtgcagagcggcctagacctcttctccatgctgctggagcagaacgacctggagcccgggcacaccgagctcctgcgcgagctgctcgcctccctgcggcgccacgacctgctgcggcgcgtcgacgacttcgaggcgggggcggcggccggggccgcgcctggggaagaagacctgtgtgcagcatttaacgtcatatgtgataatgtggggaaagattggagaaggctggctcgtcagctcaaagtctcagacaccaagatcgacagcatcgaggacagatacccccgcaacctgacagagcgtgtgcgggagtcactgagaatctggaagaacacagagaaggagaacgcaacagtggcccacctggtgggggctctcaggtcctgccagatgaacctggtggctgacctggtacaagaggttcagcaggcccgtgacctccagaacaggagtggggccatgtccccgatgtcatggaactcagacgcatctacctccgaagcgtcctga

SEQ ID NO: 17 - protein, 471 aa, synthetic construct
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSK

SEQ ID NO: 18 - RNA, 1416 nt, synthetic construct
  source 1..1416: mol_type = other RNA
atggaaaatttgaagcatattatcacccttggccaggtcatccacaaacggtgtgaagagatgaaatactgcaagaaacagtgccggcgcctgggccaccgcgtcctcggcctgatcaagcctctggagatgctccaggaccaaggaaagaggagcgtgccctctgagaagttaaccacagccatgaaccgcttcaaggctgccctggaggaggctaatggggagatagaaaagttcagcaatagatccaatatctgcaggtttctaacagcaagccaggacaaaatactcttcaaggacgtgaacaggaagctgagtgatgtctggaaggagctctcgctgttacttcaggttgagcaacgcatgcctgtttcacccataagccaaggagcgtcctgggcacaggaagatcagcaggatgcagacgaagacaggcgagctttccagatgctaagaagagataatgaaaaaatagaagcttcactgagacgattagaaatcaacatgaaagaaatcaaggaaactttgaggcagtatttaccaccaaaatgcatgcaggagatcccgcaagagcaaatcaaggagatcaagaaggagcagctttcaggatccccgtggattctgctaagggaaaatgaagtcagcacactttataaaggagaataccacagagctccagtggccataaaagtattcaaaaaactccaggctggcagcattgcaatagtgaggcagactttcaataaggagatcaaaaccatgaagaaattcgaatctcccaacatcctgcgtatatttgggatttgcattgatgaaacagtgactccgcctcaattctccattgtcatggagtactgtgaactcgggaccctgagggagctgttggatagggaaaaagacctcacacttggcaagcgcatggtcctagtcctgggggcagcccgaggcctataccggctacaccattcagaagcacctgaactccacggaaaaatcagaagctcaaacttcctggtaactcaaggctaccaagtgaagcttgcaggatttgagttgaggaaaacacagacttccatgagtttgggaactacgagagaaaagacagacagagtcaaatctacagcatatctctcacctcaggaactggaagatgtattttatcaatatgatgtaaagtctgaaatatacagctttggaatcgtcctctgggaaatcgccactggagatatcccgtttcaaggctgtaattctgagaagatccgcaagctggtggctgtgaagcggcagcaggagccactgggtgaagactgcccttcagagctgcgggagatcattgatgagtgccgggcccatgatccctctgtgcggccctctgtggatgaaatcttaaagaaactctccaccttttctaagtag

SEQ ID NO: 19 - RNA, 52 nt, synthetic construct
  source 1..52: mol_type = other RNA
gaataaactagtattcttctggtccccacagactcagagagaacccgccacc

SEQ ID NO: 20 - RNA, 105 nt, synthetic construct
  source 1..105: mol_type = other RNA
cgggtggcatccctgtgacccctccccagtgcctctcctggccctggaagttgccactccagtgcccaccagccttgtcctaataaaattaagttgcatcaagct

SEQ ID NO: 21 - RNA, 1647 nt, synthetic construct
  source 1..1647: mol_type = other RNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggatctggcggaggaagcggaggcggaggttctggcggtggtggaagcggtggcggctctggtggaagaaatctgcctgtggccactcctgatcctggcatgttcccttgtctgcaccactctcagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctaa

SEQ ID NO: 22 - RNA, 1647 nt, synthetic construct
  source 1..1647: mol_type = other RNA
atgtgtcaccagcagctggtgatcagctggttcagcttggtgttcttggccagcccactggtggccatctgggagctgaagaaggacgtgtacgtcgttgagctggactggtaccccgacgctccaggggagatggtggtgctgacctgcgacaccccggaggaggacggcatcacctggaccctggaccagtccagcgaagtgctgggcagcgggaagaccttgaccatccaggtcaaggagttcggcgacgctggccagtacacctgccacaaaggcggggaggtgctgagccacagcctgctgctgttgcacaagaaggaggacggcatctggtccacagacattctgaaggaccagaaggagccgaagaacaagaccttcctgcggtgtgaggccaagaactacagcggcaggttcacctgctggtggctgaccaccatcagcactgacctgaccttcagcgtcaagagctccaggggcagcagcgacccccagggtgtcacttgtggcgccgccaccctctcagccgagagggtgcgcggcgacaacaaggagtacgagtactccgtggagtgtcaggaggactccgcctgccccgccgccgaggagtccctgccgatcgaggtcatggtggacgccgtccacaagctgaagtatgagaattatacttccagcttcttcattcgggacatcatcaagcccgaccctcccaagaacctgcagctgaagcccctgaagaacagcaggcaggtggaggtgagctgggagtacccggacacctggagcaccccccattcttacttcagcttgaccttttgcgtgcaggtgcagggcaagagcaagcgggagaagaaggacagggtgttcacagacaagacgagcgccacggtgatctgtcgcaagaatgcgtcgatcagcgtgcgcgctcaggacaggtactacagctcaagctggtccgagtgggccagcgtcccctgcagcggcggctcagggggcggctccggcggcgggggctccggcggcggcggcagcggagggggtagcggcgggaggaaccttcccgtcgctacccccgaccccggcatgtttccttgcctgcaccacagccagaacctgctgcgggcggtaagtaacatgctgcagaaggccaggcagaccctggaattctacccctgcacctccgaggagatcgaccacgaggatatcaccaaagataagacctccaccgtggaggcgtgcctgcccctggagctcaccaagaacgagtcttgcctcaacagcagggaaacctccttcatcaccaacggcagctgcttggccagcaggaagaccagcttcatgatggccctttgtctgagctccatctacgaggatctgaagatgtatcaggtggagttcaagacaatgaacgccaagctgttgatggaccccaagaggcagatcttcttggatcagaatatgctggccgttatcgatgagctgatgcaggccctgaacttcaattctgaaaccgtgccgcagaagtccagcttagaggagcccgacttctacaagaccaagattaagctgtgcattctgctgcacgctttcagaatcagggccgtcaccatcgatagggtcatgtcatatctgaacgccagctga

SEQ ID NO: 23 - RNA, 1647 nt, synthetic construct
  source 1..1647: mol_type = other RNA
atgtgccaccagcagctggtgatcagctggttctcgctggtcttcctcgcgtcccctctggtagcgatctgggagctcaagaaggatgtctacgtggtcgagctggactggtaccctgacgccccgggggagatggtggtcttgacctgcgacaccccagaggaggacggcatcacctggactcttgaccagagctcagaggttctgggctctggcaagaccttgaccatccaggtcaaggagttcggggatgccggccagtacacctgccacaagggcggggaggtgctgagccactccctcctccttctgcacaagaaggaggacgggatctggtccacagacattctgaaggaccagaaggagcccaagaacaagaccttcctgaggtgcgaggccaagaactactccggcaggttcacctgctggtggttgaccaccatctccaccgacttgaccttctcggtcaagtcgtctaggggtagcagcgacccgcagggcgtgacctgcggggctgctacccttagcgccgagcgggtgcggggcgacaacaaggagtacgagtacagcgtagagtgccaggaggactctgcctgtcccgccgccgaggagtccctgccgatcgaggtcatggtggacgccgtgcacaagctgaagtatgagaattataccagctccttcttcatccgcgacatcatcaagcctgacccgcccaagaacctgcagctgaagccgctgaagaacagccgtcaggttgaagtgagctgggagtacccagacacctggagtactccccacagctacttcagcctgaccttctgcgtgcaggttcagggcaagtcaaagcgggagaagaaggacagggtgttcaccgacaagacctcggccaccgtgatctgcaggaagaacgctagcatttctgtgcgggcccaggaccgctactacagtagtagctggtccgagtgggccagcgttccctgcagcggtggcagcggcgggggctccggcggcggcgggagtggaggcgggggaagcggcggggggtcgggcggtaggaacctgccggttgctacccccgaccccggcatgttcccctgcctccaccactcgcagaaccttctgcgagctgtcagcaacatgctgcagaaggccaggcagaccctggaattctacccttgcaccagcgaggagatcgaccacgaagacatcactaaagacaagacctccaccgtggaggcttgtcttcctcttgagctcaccaagaacgagagctgcctgaactcgagggaaaccagcttcatcactaacgggagctgtctggcgtccaggaagactagcttcatgatggctctgtgcctgagctccatctacgaggacttgaagatgtaccaggtggagttcaagaccatgaacgcaaagctgctcatggatcccaagaggcagatcttcctggaccagaacatgctcgccgttattgatgagctcatgcaggccctgaacttcaactccgaaaccgtgccgcagaagtccagcttggaggagcccgacttctacaagaccaagatcaagctgtgcattctgctgcacgcgtttcggatcagggccgtcaccatcgacagggtgatgagctacctgaacgccagctga

SEQ ID NO: 24 - RNA, 1647 nt, synthetic construct
  source 1..1647: mol_type = other RNA
atgtgccatcagcaactggttatcagctggttcagcttggtgtttctggctagccccctggtggccatctgggagctgaaaaaggacgtgtacgtggtggagctggattggtaccccgacgccccgggcgagatggtggtgcttacctgcgatacacccgaagaggatggaatcacttggacgctggaccagtccagcgaggtgctgggttccggcaagaccctcaccatacaggtgaaggagtttggcgacgccggccagtacacctgtcacaaaggcggggaggtgctgagtcatagcctgctgctccttcacaagaaggaggacggcatctggtccacagacattctgaaggaccagaaggagcccaagaacaaaacctttctgaggtgtgaagccaaaaactactccggcaggttcacctgctggtggcttacaaccatctctaccgatctgactttctcagtgaaaagctcaagggggtcaagcgacccgcagggcgtgacctgcggcgcggccaccctgagcgctgagagagtcaggggtgacaacaaggagtacgaatactccgttgagtgtcaggaggacagcgcctgcccggcagccgaggagagcctgcccatcgaggtcatggtggacgccgtgcacaagctgaagtatgaaaattataccagctctttcttcatcagggatattatcaagcctgatccacccaagaacctgcagcttaagccactgaagaacagtaggcaggttgaggtttcttgggagtaccctgatacgtggagcactccccatagctacttcagcctgaccttctgtgtgcaggtgcaaggcaagtctaagagggagaagaaagatagggtgttcaccgacaagacctctgccaccgtgatctgcaggaagaacgctagcatttccgttcgggctcaggacaggtactactctagcagctggagcgagtgggcctcagtcccttgctctggcggcagtggtggaggcagcgggggcggcggttccggcggcggcggcagcggcggtggcagcggcggcaggaacctgccggttgccacccccgatcccgggatgttcccctgcctccatcacagccagaatctattacgtgcagtgtccaacatgttgcagaaggccaggcagactcttgagttctatccttgcacgagcgaggagatcgaccacgaggacatcaccaaggacaagacaagcactgtggaggcttgtctgcctctggagctgaccaagaacgagagctgcctgaacagcagggaaacctctttcatcactaacggcagctgcctggcctctcgcaagaccagcttcatgatggcactgtgtctgagcagtatctatgaggaccttaagatgtaccaggtggagtttaagaccatgaacgccaagctgctgatggaccccaagaggcagattttcttggatcagaacatgctcgccgtcatcgacgagctcatgcaggcgctgaatttcaacagtgagacagtgcctcagaagtccagtctggaggagcctgatttttacaagacaaagatcaagctgtgcattctgctgcacgccttccgtatcagggccgtcaccatcgacagagtgatgagctacctgaacgcctcttga

SEQ ID NO: 25 - RNA, 1647 nt, synthetic construct
  source 1..1647: mol_type = other RNA
atgtgccaccagcagctggtgatcagctggttcagcttggtgttcctggccagcccactggtggccatctgggagctgaagaaggatgtctacgtggtcgagctggactggtaccccgacgccccaggggagatggtggtgctgacctgcgacaccccggaggaggacggcatcacctggaccctggaccagtccagcgaggtgctgggcagcgggaagaccctcaccatccaggtgaaggagttcggcgacgccggccagtacacttgccacaagggtggcgaggtgctgagccactccctgctgctgcttcacaagaaggaggacggtatctggtccacagacattctgaaggaccagaaggagcccaagaacaagaccttcctgaggtgcgaggccaagaactactccggcaggttcacctgctggtggctgaccaccatcagtaccgatctgaccttctctgtgaagtccagcagggggagcagtgacccccagggggtcacctgcggcgccgccactctgagcgccgagagggtgaggggggacaacaaggagtacgagtactccgtggagtgccaggaggactccgcctgtcccgccgccgaggagtccctgccaatcgaggtcatggtggacgccgtgcacaagctgaagtacgagaactacaccagctccttcttcatccgcgacatcatcaagccagacccccccaagaacctgcagctgaagcctctgaagaacagcaggcaggttgaggtgagctgggagtacccagacacctggagtactccccacagctacttcagcctgaccttctgcgtgcaggttcaggggaagtctaagcgggagaagaaggacagggtgttcaccgacaagacctcggccaccgtcatctgcaggaagaatgcgtccatctccgtgagggcgcaggacagatactactcctcctcctggagcgagtgggcatctgtcccctgcagtggcgggagcggcgggggctccggcggcggcgggtcaggcggaggtggcagcggcggggggagcgggggcaggaacctgcccgtcgccacccccgaccctggcatgttcccctgcctgcaccactcccagaatctgctgcgggccgtgagcaacatgctccagaaggcccggcagactctggagttctatccttgcaccagcgaggagatcgaccacgaggacatcactaaagacaagacctccaccgtggaggcttgtctgcccttggagctgaccaagaacgagtcttgcctgaatagccgggaaaccagcttcatcaccaacggaagctgcctggctagcaggaagaccagcttcatgatggccctgtgcttgagctccatctacgaggacctgaagatgtaccaggtggagttcaagaccatgaacgccaagctgctgatggaccccaagcggcagatcttcctggatcagaacatgctcgccgtgatcgatgagctgatgcaggccctgaacttcaattctgaaaccgtgccgcagaagtccagcttggaggagcccgacttctacaagaccaagatcaagctgtgcattctgctgcacgctttcagaatcagggccgtcaccatcgacagggtgatgagctacctgaacgccagctga

SEQ ID NO: 26 - RNA, 1647 nt, synthetic construct
  source 1..1647: mol_type = other RNA
atgtgccaccagcagctggtgatcagctggttcagcctggtgttcctggcgagccctctggtggcaatctgggagttaaagaaggacgtgtacgtggtggagctggactggtaccccgatgcccccggcgagatggtggtactgacctgcgacacgcccgaggaggacggcatcacctggaccctggatcagagcagcgaggtgctgggtagcggcaagacccttaccattcaggtgaaggagttcggcgacgccggccagtacacttgccacaagggtggcgaggtgctgagccattctctgctgcttcttcacaagaaagaggacggtatctggtccaccgacatcctgaaggatcagaaggagcctaagaacaagacctttctgcggtgcgaggccaagaattactcaggcaggttcacctgctggtggctgactaccatcagcaccgacctgactttctctgtgaagtccagcaggggaagcagtgacccccagggggttacctgcggcgccgccacccttagcgccgaaagggtgaggggcgataacaaggagtacgaatactctgtggagtgtcaggaggacagcgcctgtccagctgctgaggagagcttgcctattgaggttatggtggacgccgtccacaagctgaagtatgagaattataccagctcgttcttcatcagggacataatcaaacctgatccccctaagaacttgcagctgaagccactaaagaacagcaggcaggttgaggtaagctgggagtacccagacacctggagtactccgcacagctacttcagcttgaccttctgtgtgcaggttcagggcaagtctaagagggagaagaaggacagggtgttcaccgacaagacctcagccaccgtgatctgcaggaagaacgcaagcatcagcgtgagggcccaggacaggtactactcaagcagctggagcgagtgggccagcgtcccctgcagcggcggcagcggcggcggtagcggcggcgggggtagcggcggcggcgggtctggcggcggcagcggcggcagaaacctgcccgtcgctacccccgaccccggtatgttcccttgcttgcaccactctcagaatctgcttagggccgtgagcaatatgctgcagaaggccaggcagactctggagttctacccgtgtacgagcgaggagatcgatcacgaggacatcactaaggacaaaacctctaccgtcgaggcctgcctgccgctggagctgaccaagaacgaatcttgcctgaactccagggaaaccagcttcatcacgaacggcagctgcctggccagcaggaagacctctttcatgatggccctgtgcctgagcagcatctatgaggaccttaagatgtatcaggtggagtttaagaccatgaacgccaagctgctgatggaccccaaacgccagatatttctggatcagaacatgctggccgtgattgatgagctgatgcaagcccttaactttaactccgaaaccgtgccccagaagagttcgctagaggagcccgatttttacaagaccaagatcaaactgtgcatcctgctgcacgcgtttaggatcagggctgtgacaatcgaccgggtgatgagctacctgaacgccagctaa

SEQ ID NO: 27 - RNA, 552 nt, synthetic construct
  source 1..552: mol_type = other RNA
atggaaagagtgcagcccctggaagagaacgtgggcaatgccgccagacctagattcgagcggaacaagctgctgctggtggcctctgtgatccaaggactgggactgctgctgtgcttcacctacatctgcctgcacttcagcgccctccaggtgtcccacagataccccagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaatatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctgtaa

SEQ ID NO: 28 - RNA, 552 nt, synthetic construct
  source 1..552: mol_type = other RNA
atggagcgggttcagcccctggaggagaatgtaggtaacgcagccaggcccaggttcgagcggaacaagctgctgcttgtagcttccgtgattcagggcctgggcctgctgctgtgttttacctacatttgcctccatttcagcgctctgcaggtgtcacacaggtacccaaggatccagtccatcaaagtgcagttcacggagtacaagaaggagaagggcttcatcctcacctctcagaaggaagatgagatcatgaaggtgcagaacaactccgtgatcatcaactgcgacggcttctacctcatctctctgaagggctactttagccaggaggttaacattagcctccactatcagaaggatgaggagccgttgttccagctgaagaaggtgaggtccgtgaactctttgatggtggcatccttgacctacaaggacaaggtgtacctgaatgtgaccaccgacaacacgagcctggatgacttccatgtgaatggaggggagctgatcctgatccatcagaacccaggggagttctgcgtcctctga

SEQ ID NO: 29 - RNA, 552 nt, synthetic construct
  source 1..552: mol_type = other RNA
atggagagggtgcagcccctggaggagaacgttggcaacgccgcgaggcccaggttcgagcgcaacaagctcctcctggttgccagcgtgatccaggggctggggctgctgttgtgctttacatacatttgtttgcacttcagcgccctgcaggtgtcacacaggtaccctcgtatccagagtattaaggtgcagttcaccgagtacaagaaggagaagggcttcatcttgacctcccagaaggaggacgagataatgaaggtccagaacaactccgtaatcatcaactgtgacggattttaccttatcagcttgaaggggtacttctcccaggaggtgaacatcagcctccactaccagaaggacgaggagccccttttccagctgaagaaggtgagatccgtcaacagtctgatggttgcgagtctgacctacaaggataaggtgtacctgaatgtgaccaccgacaacacctctctcgatgactttcacgtgaacgggggcgagctgattctgatccatcagaatcccggcgagttctgcgttctgtga

SEQ ID NO: 30 - RNA, 552 nt, synthetic construct
  source 1..552: mol_type = other RNA
atggagagggtgcagcccctggaggagaacgtcggcaatgccgccaggcctaggtttgagaggaacaaactgctgctcgtggccagcgtgatccagggcctgggcctgctcctctgcttcacctatatctgcctgcacttcagtgcactgcaggtgtcacataggtaccctaggattcagagtatcaaggtacagttcactgagtataagaaggagaagggctttatcctcacttctcagaaggaggatgagatcatgaaggtacagaacaactcggtgatcataaactgtgacgggttttaccttatttcactgaagggctacttctctcaggaggtgaacattagtttacactaccagaaagacgaggagcccttatttcagctgaagaaggtgaggtccgttaatagtcttatggtcgccagtctgacctataaggataaggtgtacctgaacgtgacaacggacaatacctcccttgacgacttccacgttaacggcggtgaactgattttgatccaccagaatcctggcgagttctgtgtgctctaa

SEQ ID NO: 31 - RNA, 552 nt, synthetic construct
  source 1..552: mol_type = other RNA
atggagcgggtccagcccctggaggagaacgttggcaacgcagcgcggcccaggttcgagaggaacaagctgctgctggttgccagcgtgatccaggggctgggcctgctcctgtgcttcacctacatctgcctgcacttcagcgcactgcaggtgagccacaggtatcccaggatacagagcatcaaggtgcagttcaccgagtacaagaaggagaagggcttcatcctcacttcccagaaggaggatgagatcatgaaggtgcagaacaacagcgtgatcatcaactgtgacggcttctacctgatcagcctgaaggggtacttcagccaggaggtgaacatcagcctccactaccagaaggatgaggagcccctgtttcagctgaagaaggtgaggtccgtcaacagtctgatggtcgcgtccttgacctacaaggacaaggtgtacctgaacgtgaccaccgacaacaccagcctggacgacttccatgtgaacggcggtgagctgatcttgattcaccagaaccctggtgagttctgtgtcctgtga

SEQ ID NO: 32 - RNA, 552 nt, synthetic construct
  source 1..552: mol_type = other RNA
atggagagggtgcagcccctggaggagaacgtgggtaacgcggcccggccccggttcgagcgcaacaagctgctgcttgttgcgtccgtgatccaggggctgggcctgctcctgtgcttcacctacatctgcctgcacttcagcgcgttgcaggtgtcgcacaggtacccgcggatccagagcatcaaggtgcagttcacggagtacaagaaggagaagggcttcatcctcacctcccagaaggaggatgagatcatgaaggtgcagaacaacagcgtgatcatcaactgtgacggcttctacctcatcagcttgaaggggtacttctcgcaggaggtgaacatcagcctccactaccagaaggacgaggagcccctcttccagctgaagaaggtgaggtccgtcaacagcttgatggtcgcgtccttgacctacaaggacaaggtgtacctcaacgtgaccacggacaacacgtccctggacgacttccacgtgaacgggggtgagttgatactcatccaccagaacccgggggagttctgcgtcctctag

SEQ ID NO: 33 - RNA, 552 nt, synthetic construct
  source 1..552: mol_type = other RNA
atggagcgcgtgcagccgctggaggaaaacgtcgggaacgccgctcgccctaggttcgagaggaataagcttctcctggtggcgagcgtgatccagggactgggcctgttactatgtttcacctacatatgcctgcactttagcgcgttgcaggtgagccatagataccctaggatccagagtattaaggtgcagttcaccgagtacaagaaggagaagggattcatcctcaccagtcagaaggaggacgagatcatgaaggttcagaacaattccgtgattatcaactgtgatggcttctacctcatcagcctgaagggctatttcagtcaggaagtgaacatttccctgcactatcaaaaggatgaggagcccctttttcagctgaagaaggtgaggtccgttaactctctgatggtggcctctctgacctacaaggacaaggtgtacctgaacgtgaccaccgataatacttccctggatgatttccacgttaacggcggcgagctgatcctgattcaccagaatcccggcgagttttgcgtgctctag

(19) United States
(12) Patent Application Publication
(10) Pub. No.: US 2025/0177432 A1
(43) Pub. Date: Jun. 5, 2025

(54) RNA MOLECULES AND COMPOSITIONS FOR CANCER IMMUNOTHERAPY

(71) Applicant: WUHAN HOUXIAN BIOPHARMACEUTICAL CO. LTD., Wuhan (CN)

(72) Inventors: Hongbing SHU, Wuhan (CN); Mi LI, Wuhan (CN); Shu LI, Wuhan (CN)

(21) Appl. No.: 18/967,090

(22) Filed: Dec. 3, 2024

(30) Foreign Application Priority Data
Dec. 4, 2023 (WO) PCT/CN2023/136140

(51) Int. Cl. A61K 31/7105; A61K 9/00; A61K 9/127; A61K 9/51; A61P 35/00

(52) U.S. Cl. CPC A61K 31/7105; A61K 9/0019; A61K 9/1271; A61K 9/5123; A61P 35/00

(57) ABSTRACT

This disclosure provides compositions and methods for treating cancers. The compositions include mRNA molecules encoding proteins useful for treating the cancers, such as IL-12, an OX40 agonist, and optionally along with FADD, MLKL or MLKL-4HB. The mRNA can include 5′UTR and 3′UTR sequences and 5′cap structures that improve the stability and/or therapeutic effects of these mRNA molecules.

### CROSS REFERENCE TO RELATED APPLICATIONS

The present application claims priority of International Application No. PCT/CN2023/136140, filed Dec. 4, 2023, the content of which is incorporated herein by reference in its entirety.

### REFERENCE TO AN ELECTRONIC SEQUENCE LISTING

The contents of the electronic sequence listing (379409.xml; Size: 52,352 bytes; and Date of Creation: Dec. 3, 2024) is herein incorporated by reference in its entirety.

### BACKGROUND

Cancer immunotherapies, such as checkpoint inhibitors and adoptive cell therapy, manipulate the immune system to recognize and attack cancer cells. An example is to enhance the effector function of tumor-specific Teff cells, and another is to reduce the inhibitory function of tumor-specific Treg cells.

A typical cancer immunotherapy uses an antibody that targets a checkpoint protein or a tumor-associated antigen, or the immune cells engineered to express a targeting receptor. Antibodies, however, generally have limited usage, and cell therapies are extremely expensive to manufacture.

Therapeutic agents, such as proteins, may also be delivered as encoding mRNA, which is an emerging technology. mRNA molecules, however, tend to be unstable and more immunogenic. Further developments are needed to optimize these technologies.

### SUMMARY

It is demonstrated herein that certain structural elements can help improve the stability and/or therapeutic activity of mRNA molecules. For instance, compared to other untranslated regions (UTR), the combination of Group H with a 5′UTR of SEQ ID NO:19 and a 3′UTR of SEQ ID NO:20 achieved higher translation levels. Also, among different 5′cap structures, Cap1 appeared to provide the best antitumor effects for the mRNA. Such structural elements can be included in mRNA molecules that encode therapeutic proteins, such as IL-12 and OX40 agonist, and optionally along with FADD or MLKL (or the four-helix bundle domain, MLKL-4HB).

One embodiment of the present disclosure provides a message RNA (mRNA), comprising a 5′ untranslated region (UTR), a 3′UTR and a coding sequence, wherein the 5′UTR comprises the nucleic acid sequence of SEQ ID NO:19 and the 3′UTR comprises the nucleic acid sequence of SEQ ID NO:20.

In some embodiments, the mRNA comprises a Cap-1 structure at the 5′ end. In some embodiments, the Cap-1 structure is formed by a CleanCap® co-transcriptional capping reagent.

In some embodiments, wherein the mRNA is transcribed by a T7 RNA polymerase. In some embodiments, the mRNA further comprises a poly(A) tail, which is preferably prepared by an enzymatic method or a co-transcriptional method. In some embodiments, the poly(A) comprises at least 50 adenine bases, preferably at least 80 adenine bases, more preferably at least 100 adenine bases.

In some embodiments, the coding sequence encodes IL-12 or an OX40 agonist or both an IL-12 and an OX40 agonist. In some embodiments, the OX40 agonist is an OX40 ligand (OX40L), a polypeptide comprising the extracellular domain of OX40L, or an agonist anti-OX40 antibody or antigen-binding fragment thereof. In some embodiments, the coding sequence further encodes FADD (FAS-associated death domain protein), MLKL (mixed lineage kinase domain like pseudokinase) or the four-helix bundle domain, MLKL-4HB.

In some embodiments, the coding sequence encoding IL-12 comprises a nucleic acid sequence selected from the group consisting of SEQ ID NO:21-26. In some embodiments, the coding sequence encoding OX40L comprises a nucleic acid sequence selected from the group consisting of SEQ ID NO:27-33, preferably selected from the group consisting of SEQ ID NO: 27, 28, 30, 32 and 33.

Also provided, in one embodiment, is a pharmaceutical composition comprising the mRNA, and an ionizable lipid, a phospholipid, a structural lipid, a polyethylene glycol (PEG) lipid, or a combination thereof. In some embodiments, the ionizable lipid is SM102 or ALC0315.

Also provided is a method for treating cancer in a patient, comprising administering to the patient the mRNA or the pharmaceutical composition of the present disclosure.

Another embodiment provides a method for treating cancer in a patient, comprising administering to the patient a first mRNA encoding an OX40 agonist, a second mRNA encoding IL-12, and a third mRNA encoding FADD or MLKL (or the four-helix bundle domain, MLKL-4HB).

In some embodiments, the third mRNA is on the same RNA molecule as the first mRNA or the second mRNA. In some embodiments, the OX40 agonist is an OX40 ligand (OX40L), a polypeptide comprising the extracellular domain of OX40L, or an agonist anti-OX40 antibody or antigen-binding fragment thereof.

In some embodiments, the first mRNA and the second mRNA are administered at a mass ratio of 4:1 to 0.5:1, preferably 3:1 to 0.75:1, more preferably 2:1 to 0.9:1.

In some embodiments, the administration is subcutaneous injection, intramuscular injection, intraperitoneal injection, thoracic injection, intravenous injection, arterial injection, or a combination thereof.

In some embodiments, the cancer is selected from the group consisting of squamous cell carcinoma, lung cancer, peritoneal cancer, hepatocellular carcinoma, gastric cancer, pancreatic cancer, glioblastoma, cervical cancer, ovarian cancer, liver cancer, bladder cancer, urethral cancer, breast cancer, colon cancer, rectal cancer, colorectal cancer, endometrial cancer, uterine cancer, salivary gland cancer, kidney cancer, prostate cancer, vulvar cancer, thyroid cancer, liver cancer, anal cancer, soft tissue sarcoma, neuroblastoma, penile cancer, melanoma, superficial spreading melanoma, lentigines melanoma, acral melanoma, nodular melanoma, multiple bone marrow tumor, B-cell lymphoma, chronic lymphocytic leukemia, non-Hodgkin's lymphoma, acute lymphoblastic leukemia, hairy cell leukemia, chronic myeloblastic leukemia, post-transplant lymphoproliferative disorder, brain tumor, and brain cancer and head and neck cancer, preferably colon cancer, breast cancer and lung cancer.

Further provided is a pharmaceutical composition, comprising a pharmaceutical composition, comprising a first mRNA encoding an OX40 agonist and/or IL-12, and a second mRNA encoding FADD or MLKL (or the four-helix bundle domain, MLKL-4HB). In some embodiments, the first mRNA encodes the OX40 agonist and the second mRNA encodes FADD or MLKL (or MLKL-4HB), wherein the composition optionally further comprises a third mRNA encoding IL-12. In some embodiments, the first mRNA encodes the IL-12 and the second mRNA encodes FADD or MLKL (or MLKL-4HB), wherein the composition optionally further comprises a third mRNA encoding an OX40 agonist.

Still further provided, in one embodiment, is an mRNA comprising a nucleic acid sequence selected from the group consisting of SEQ ID NO:21-26. Also provided is an mRNA comprising a nucleic acid sequence selected from the group consisting of SEQ ID NO:27-33, preferably selected from the group consisting of SEQ ID NO:27, 28, 30, 32 and 33.

### BRIEF DESCRIPTION OF THE DRAWINGS

FIG. 1 shows the results of screening for single or combination mRNA molecules having anti-tumor effects in a colon cancer animal model.

FIG. 2 shows that the therapeutic effects of the combination of OX40L and IL-12 mRNA spread from the injection side to the distal tumor side.

FIG. 3 shows tumor mass reduction in animals following intratumoral injection of mRNA expressing the test agents.

FIG. 4 shows overall body weight changes in animals following intratumoral injection of mRNA expressing the test agents.

FIG. 5 shows tumor volumes at the injected side, on days 8, 11, 14 and 17 post-implantation, after two intratumoral injections of the indicated mRNAs at the indicated dosages.

FIG. 6 shows tumor volumes at the distal side, on days 8, 11, 14 and 17 post-implantation, after two intratumoral injections of the indicated mRNAs at the indicated dosages.

FIG. 7 shows tumor mass reduction in animals following intratumoral injection of test mRNA molecules or their combinations.

FIG. 8 shows overall body weight changes in animals following intratumoral injection of the indicated mRNA.

FIG. 9 shows tumor volumes at the injected side, on days 9, 12, 15 and 18 post-implantation, after two intratumoral injections of the indicated mRNA.

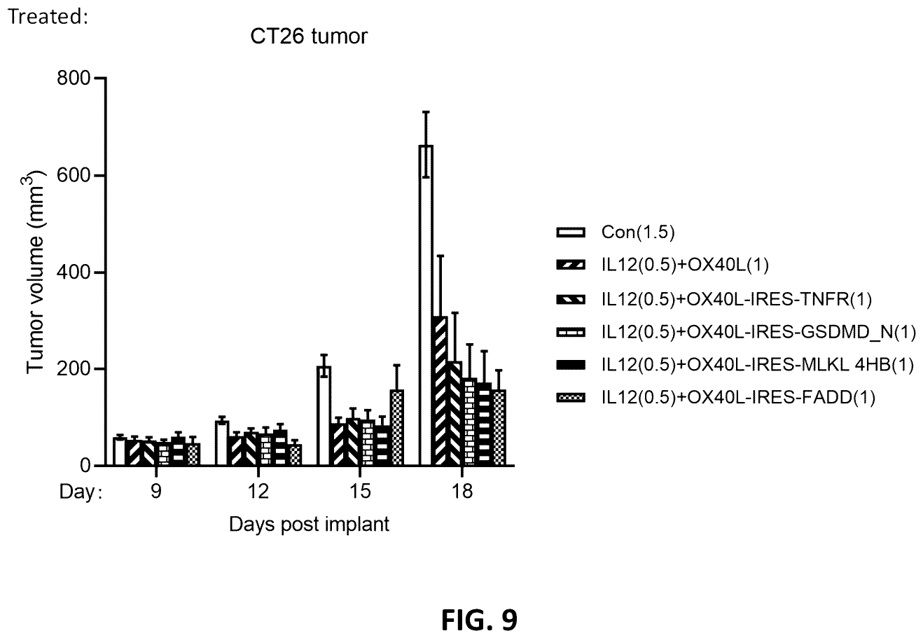

FIG. 10 shows tumor volumes at the distal side, on days 9, 12, 15 and 18 post-implantation, after two intratumoral injections of the indicated mRNA.

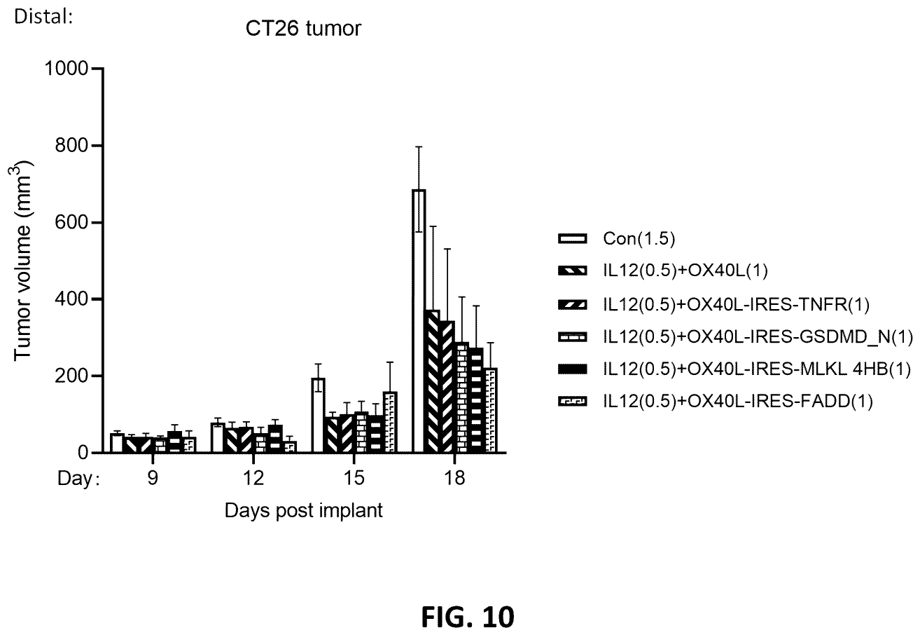

FIG. 11 shows anti-tumor effects of OX40L and IL-12 mRNA delivered with different ionizable lipids.

FIG. 12 shows distributions of luciferase mRNA delivered with different ionizable lipids.

FIG. 13 shows anti-tumor effects of OX40L and IL-12 mRNA produced with different RNA polymerases and capping methods.

FIG. 14 shows translation of EGFP mRNA transcribed by T7 or VSW-3 RNA polymerase.

FIG. 15 shows anti-tumor effects of OX40L and IL-12 mRNA with different cap structures.

FIG. 16 shows translation of EGFP mRNA with different UTR combinations.

FIG. 17 shows translation of OX40L and IL-12 mRNA with codon optimization.

FIG. 18 shows translation of EGFP mRNA with different poly(A) lengths.

### DETAILED DESCRIPTION

### Definitions

It is to be noted that the term “a” or “an” entity refers to one or more of that entity; for example, “an mRNA,” is understood to represent one or more mRNA. As such, the terms “a” (or “an”), “one or more,” and “at least one” can be used interchangeably herein.

OX40, also known as CD134 and tumor necrosis factor receptor superfamily, member 4 (TNFRSF4), is a member of the TNFR-superfamily of receptors. Unlike CD28 which is constitutively expressed on resting naïve T cells, OX40 is a secondary co-stimulatory immune checkpoint molecule, expressed after 24 to 72 hours following activation.

OX40L, also known as CD252, is the ligand for OX40 and is stably expressed on many antigen-presenting cells such as DC2s (a subtype of dendritic cells), macrophages, and activated B lymphocytes. OX40L is also present on the surface of many non-immune cells, such as the endothelial cells and the smooth muscle cells. The surface expression of OX40L can be induced by many pro-inflammatory mediators, such as TNF-α, IFN-γ, and PGE2 (Prostaglandin E2).

A representative nucleic acid sequence for human OX40L (isoform 1) is provided in NCBI Reference No. NM_003326 with a corresponding protein sequence in NP_003317. Another representative nucleic acid sequence for human OX40L (isoform 2) is provided in NCBI Reference No. NM_001297562 with a corresponding protein sequence in NP_001284491. Isoform 1 has a longer N-terminus than isoform 2, but otherwise they are identical.

Interleukin 12 (IL-12) is an interleukin that is naturally produced by dendritic cells, macrophages, neutrophils, and human B-lymphoblastoid cells (NC-37) in response to antigenic stimulation. IL12 is a heterodimeric cytokine encoded by two separate genes, IL12A (p35) and IL12B (p40).

IL-12 is involved in the differentiation of naive T cells into Th1 cells. It stimulates the production of interferon-gamma (IFN-γ) and tumor necrosis factor-alpha (TNF-α) from T cells and natural killer (NK) cells, and reduces IL-4 mediated suppression of IFN-γ. IL-12 plays an important role in the activities of natural killer cells and T lymphocytes. IL-12 mediates enhancement of the cytotoxic activity of NK cells and CD8+ cytotoxic T lymphocytes.

IL-12 binds to the IL-12 receptor, which is a heterodimeric receptor formed by IL-12Rβ1 and IL-12Rβ2. Upon binding, IL-12R-β2 becomes tyrosine phosphorylated and provides binding sites for kinases, Tyk2 and Jak2.

A representative nucleic acid sequence for human IL-12A (isoform 1) is provided in NCBI Reference No. NM_000882 with a corresponding protein sequence in NP_000873. Another representative nucleic acid sequence for human IL-12A (isoform 2) is provided in NCBI Reference No. NM_001354582 with a corresponding protein sequence in NP_001341511. Another representative nucleic acid sequence for human IL-12A (isoform 3) is provided in NCBI Reference No. NM_001354583 with a corresponding protein sequence in NP_001341512. Another representative nucleic acid sequence for human IL-12A (isoform 4) is provided in NCBI Reference No. NM_001397992 with a corresponding protein sequence in NP_001384921.

A representative nucleic acid sequence for human IL-12B is provided in NCBI Reference No. NM_002187 with a corresponding protein sequence in NP_002178.

FAS-associated death domain protein (FADD), also called MORT1, is an adaptor protein that bridges members of the tumor necrosis factor receptor superfamily, such as the Fas-receptor, to procaspases 8 and 10 to form the death-inducing signaling complex (DISC) during apoptosis. FADD has also been seen to play a role in other processes including proliferation, cell cycle regulation and development.

A representative nucleic acid sequence for human FADD is provided in NCBI Reference No. NM_003824 with a corresponding protein sequence in NP_003815.

Mixed lineage kinase domain like pseudokinase (MLKL) plays a role in tumor necrosis factor (TNF)-induced necroptosis, a programmed cell death process, via interaction with receptor-interacting protein 3 (RIP3), which is a key signaling molecule in necroptosis pathway.

A representative nucleic acid sequence for human MLKL is provided in NCBI Reference No. NM_152649 with a corresponding protein sequence in NP_689862.

Improved mRNA Structures and Formulations

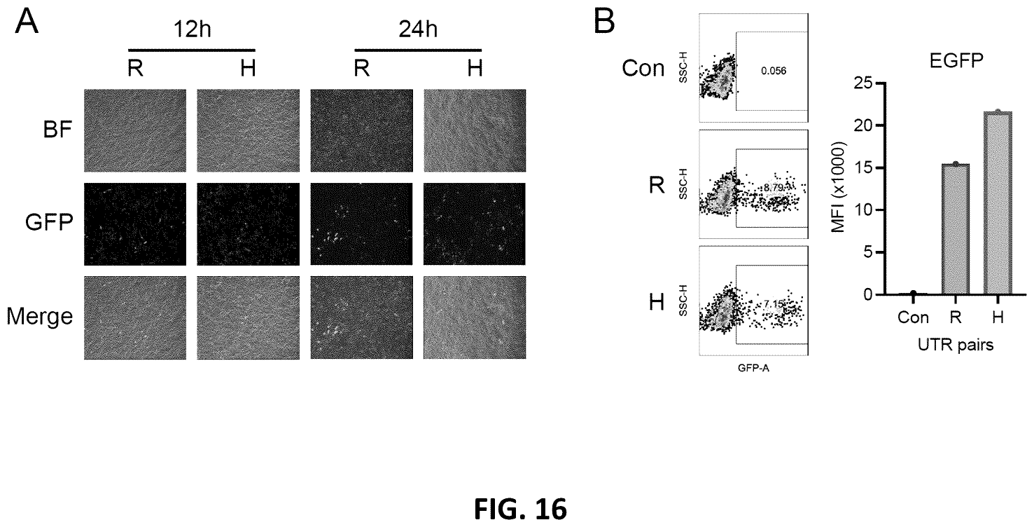

Through testing and comparative studies, the instant inventors have discovered that certain structural elements can help improve the stability, translation, and/or overall antitumor efficacy of mRNA molecules. In one example, a 5′UTR/3′UTR combination, referred to as “group H,” led to the highest translation levels when included in a mRNA molecule, flanking the coding sequence (FIG. 16). As shown in Table 1, group H includes a 5′UTR of SEQ ID NO: 19 and a 3′UTR of SEQ ID NO:20.

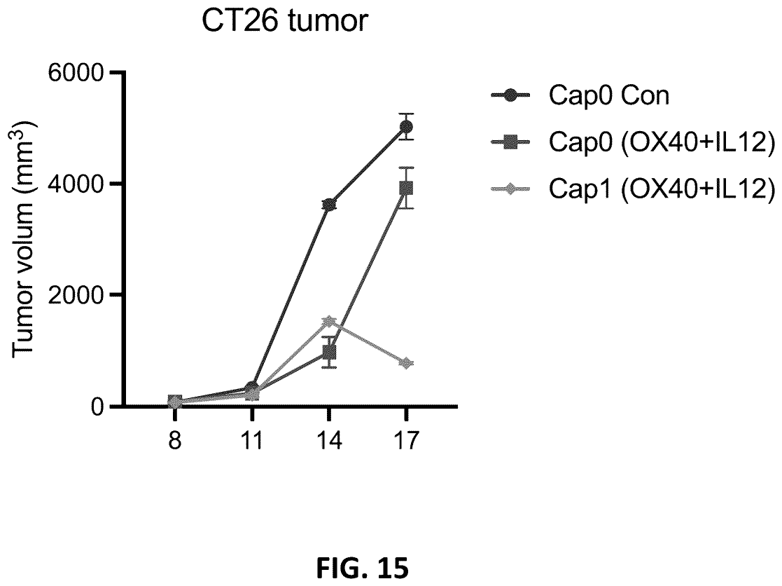

The transcription and capping methods also have impact on the stability and efficacy of the mRNA. As shown in FIG. 15, the Cap-1 structure produced a more sustainable antitumor effect than the Cap-0 structure. Also, interestingly, the CleanCap® Reagent AG system that forms the Cap-1 structure was not compatible with the VSW-3 RNA polymerase using the optimal IVT conditions (see, e.g., Xia, H. et al., (2022). RNA Biol 19, 1130-1142. 10.1080/15476286.2022.2139113). Therefore, the T7 RNA polymerase is a better transcription system for the CleanCap® Reagent AG system.

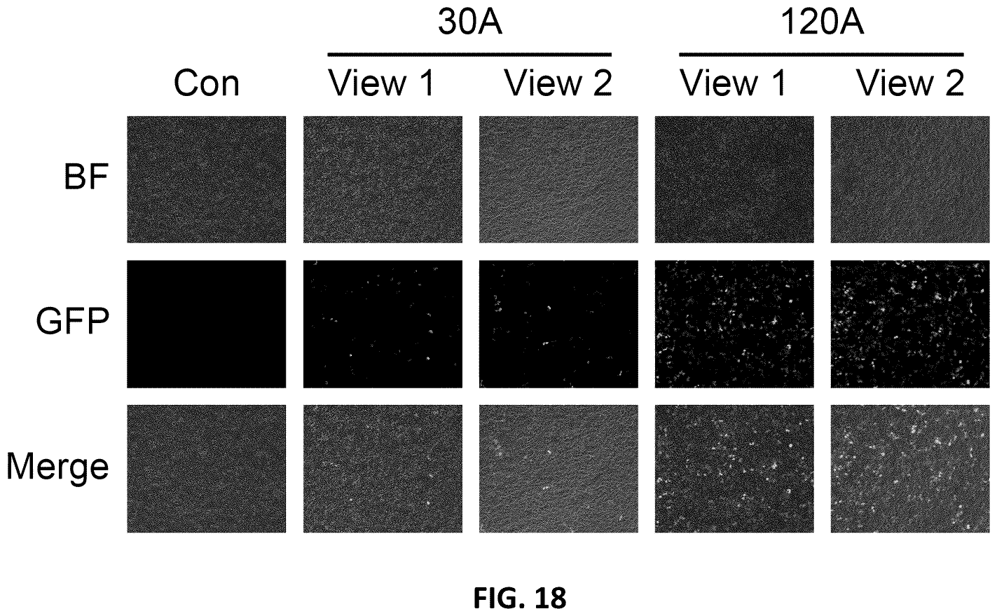

Another structural element is the poly(A) tail. As shown in FIG. 18, longer poly(A) (e.g., 120A) resulted in higher translation than the shorter one (e.g., 30A).

Various lipid nanoparticle formulations were tested for the mRNA molecules of the present disclosure. Both tested ionizable lipids, SM102 and ALC0315, provided high quality formulations. It appeared that while SM102 led to longer exposure of the delivered mRNA, ALC0315 produced the quicker uptake.

In accordance with one embodiment of the present disclosure, therefore, provided is a mRNA molecule with one or more such structural/formulation elements. In one embodiment, provided is a message RNA (mRNA) having a 5′ untranslated region (UTR), a 3′UTR and a coding sequence. In some embodiments, the 5′UTR includes the nucleic acid sequence of SEQ ID NO: 19. In some embodiments, the 3′UTR includes the nucleic acid sequence of SEQ ID NO: 20. In some embodiments, the 5′UTR is disposed at the 5′ side of the coding sequence. In some embodiments, the 3′UTR is disposed at the 3′ side of the coding sequence.

In another embodiment, provided is a message RNA (mRNA) having a 5′ cap structure. In one embodiment, the 5′cap is Cap-0. In another embodiment, the 5′cap is Cap-1. In another embodiment, the 5′cap is Cap-2.

Cap-0 is a N7-methyl guanosine connected to the 5′ nucleotide through a 5′ to 5′ triphosphate linkage, also referred to as m7G cap or m7Gppp-. An additional methylation on the 2′O position of the initiating nucleotide generates Cap-1, or referred to as m7GpppNm-, where Nm denotes any nucleotide with a 2′O methylation. Along the same line, Cap-2 has the m7G-ppp-Nm-Nm structure, as illustrated in the structure below.

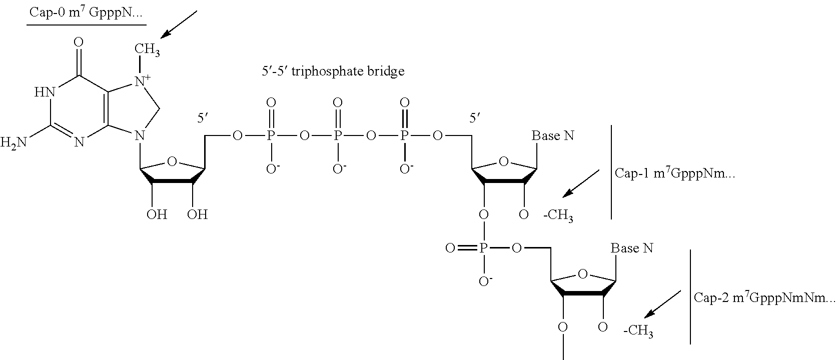

In some embodiments, the mRNA Cap-1 structure is formed by a CleanCap® co-transcriptional capping reagent, which is commercially available from TriLink Biotechnologies (San Diego, CA). In some embodiments, the mRNA is transcribed with the T7 RNA polymerase, without limitation.

In some embodiments, the mRNA further includes a poly(A) tail. In some embodiment, the poly(A) has at least 50 adenine bases, or at least 60, 70, 80, 90, 100, 110, or 120 adenine bases.

Also provided, in one embodiment, is a pharmaceutical composition comprising the mRNA of the present disclosure, and an ionizable lipid, a phospholipid, a structural lipid, a polyethylene glycol (PEG) lipid, or a combination thereof. In some embodiments, the ionizable lipid is SM102 (9-Heptadecanyl 8-{(2-hydroxyethyl) [6-oxo-6-(undecyloxy) hexyl]amino}octanoate; CAS number: 2089251-47-6) or ALC0315 ([(4-Hydroxybutyl) azanediyl]di(hexane-6,1-diyl)bis(2-hexyldecanoate); CAS number: 2036272-55-4). In some embodiments, the composition is presented as a composition of lipid nanoparticles.

The term “lipid nanoparticle” refers to particles having at least one dimension on the order of nanometers (e.g., 1-1,000 nm) which include one or more of the compounds of Formula I, or a pharmaceutically acceptable salt, stereoisomer, or mixture of stereoisomers thereof (e.g., SM102, ALC0315, or a pharmaceutically acceptable salt, stereoisomer, or mixture of stereoisomers thereof). Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or crosslinked to one another. Lipid bilayers may include one or more ligands, proteins, or channels.

As used herein, a “phospholipid” is a lipid that includes a phosphate moiety and one or more carbon chains, such as unsaturated fatty acid chains. A phospholipid may include one or more multiple (e.g., double or triple) bonds. Non-limiting examples of phospholipid include 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-OT-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanol amine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and combinations thereof. In some embodiments, the phospholipid is DOPE. In some embodiments, the phospholipid is DSPC.

In some embodiments, the composition includes a structural lipid. Non-limiting examples of structural lipid include cholesterol, fecosterol, sitosterol, ergosterol, campesterol, stigmasterol, brassicasterol, tomatidine, ursolic acid, alpha-tocopherol, and combinations thereof. In one embodiment, the structural lipid is cholesterol.

In some embodiments, the composition includes a PEG lipid. Non-limiting examples of PEG lipid include a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and combinations thereof.

### Anti-Tumor Proteins and Protein Combinations

Cancer immunotherapies have shown great promises by using small molecules, antibodies or engineered immune cells targeting numerous factors involved in the cancer-immunity cycle. A typical strategy involves activation of stimulatory factors that promote immunity, or inhibition of factors that reduce immune activity and/or prevent autoimmunity. Some prominent examples are anti-CTLA4 antibodies and anti-PD-1 or anti-PD-L1 antibodies.

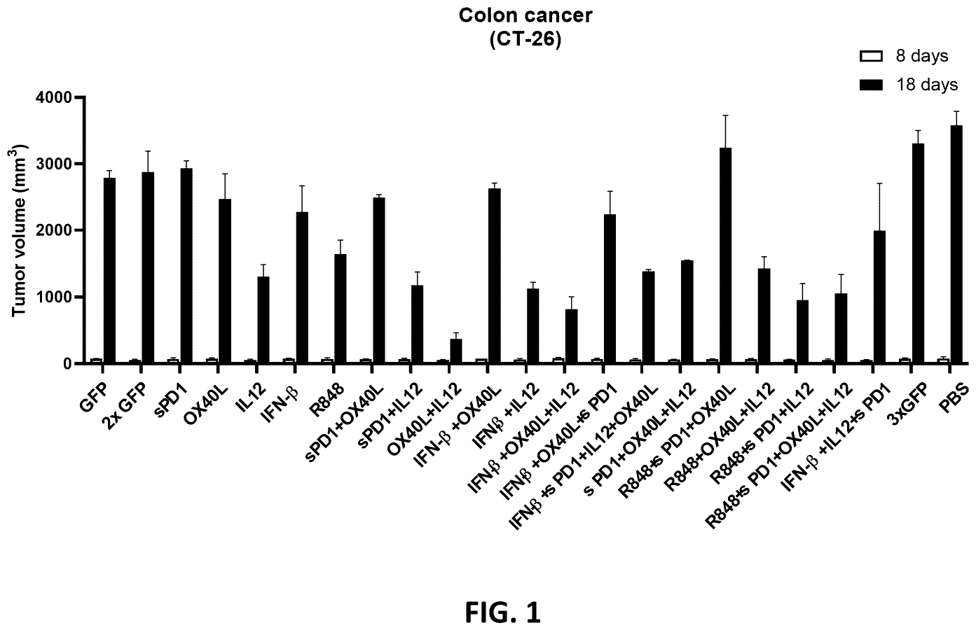

Delivery of a cancer therapeutic through an encoding mRNA is an emerging technology, which has shown some promises. There are some unforeseen obstacles, however. For instance, when an mRNA encoding a soluble PD1 fragment (sPD1) which is a known PD-1/PD-L1 inhibitor was delivered intratumorally, it exhibited no inhibition of tumor growth at all (Example 1, FIG. 1).

Also, intratumoral injection of the mRNA encoding OX40L only exhibited slight inhibition of tumor growth. By contrast, intratumoral injection of the mRNA encoding IL-12 led to marked tumor growth inhibition, at about 50% rates. (FIG. 1). Quite unexpectedly, when both mRNA were delivered to the same animal, the inhibition reached a whopping ˜90%. Such significantly enhanced therapeutic efficacy clearly indicates synergism between these molecules.

This result is unexpected in particular in view that the addition of other seemingly therapeutic mRNA molecule did not further increase efficacy. For instance, despite its moderate anti-tumor effect as a stimulator of innate immunity, the single small molecule agent R848 (a ligand for Toll-like receptor 7/8) actually decreased other agents' anti-tumor effects when used in combinations (FIG. 1).

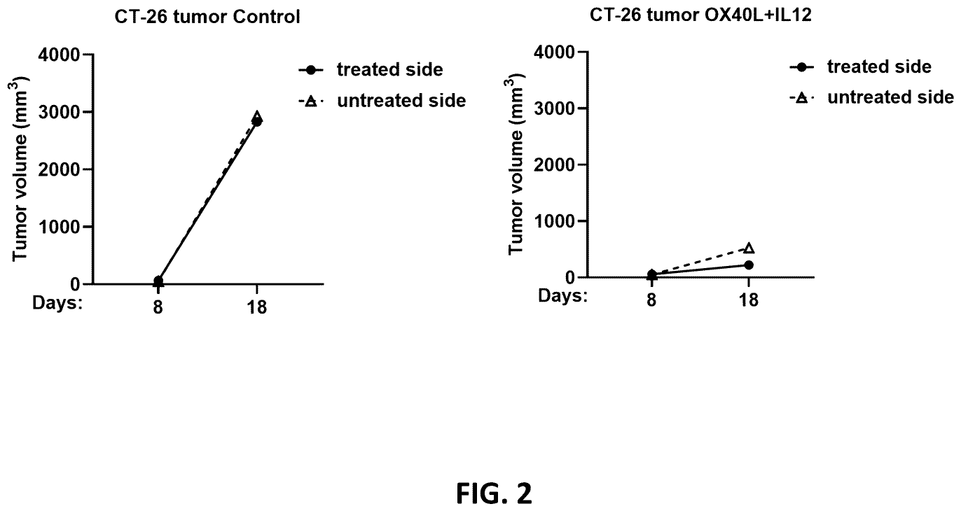

Additional experimental data presented in the examples further reinforce the therapeutic efficacy of these combination approach. As shown in FIG. 2, not only did intratumoral injection inhibit local tumor growth, but it also achieved similar magnitude of therapeutic efficacy at distal regions, across the tumor block.

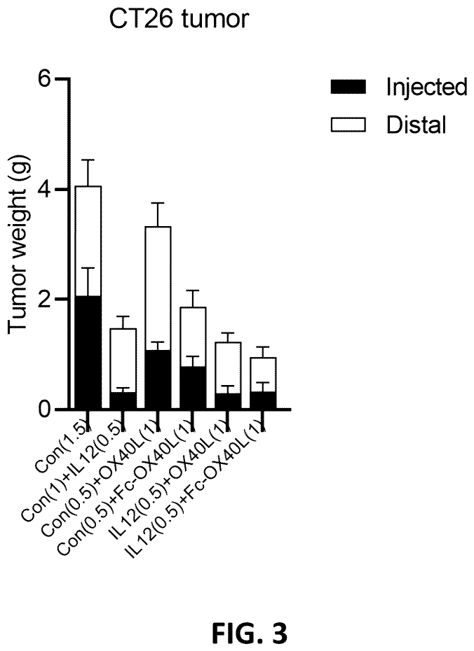

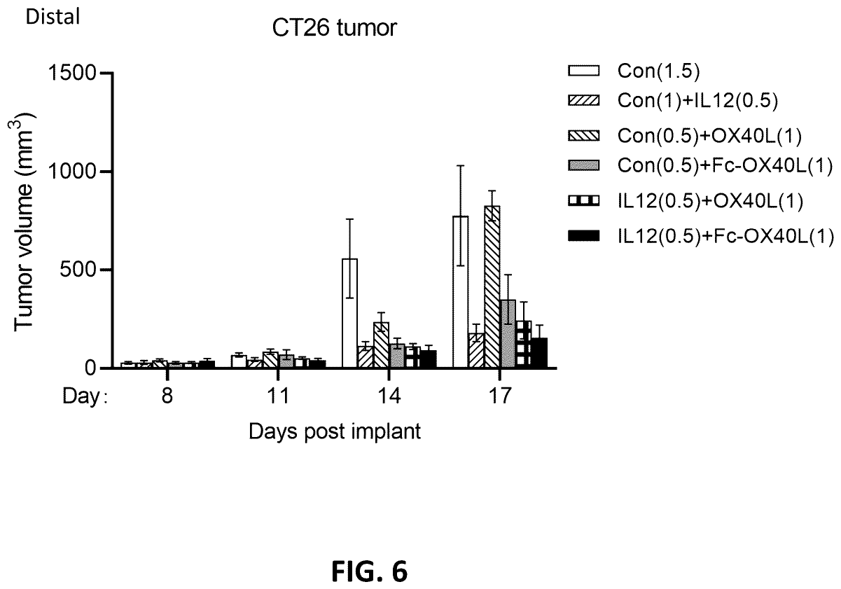

In another unexpected discovery, when a soluble counterpart of the OX40L protein was used in the combination, further improvement of the therapeutic efficacy was observed (Example 3, FIG. 3-6), in particular at the distal side of the animal, from the injection side (FIGS. 3 and 6). This soluble counterpart was an extracellular fragment of the OX40L protein fused to an IgG Fc fragment (Fc-OX40L).

In another interesting finding, when FADD or MLKL (or MLKL-4HB) was further added to the combination, the anti-tumor effects were further improved. The magnitude of improvement by FADD or MLKL (or MLKL-4HB) was greater than by GSDMD and TNFR, two other commonly used immune modulators in cancer therapies, surprisingly.

According to one embodiment of the present disclosure, therefore, provided is a method for treating cancer that entails administration of a mRNA encoding an OX40 agonist (e.g., OX40L or a protein/polypeptide/antibody or their combination that activates OX40 (e.g., an OX40L protein or anti-OX40 agonist antibody)), and an mRNA encoding IL-12 or a protein/polypeptide/antibody or their combination that acts like IL-12 to stimulate IL-12 receptor (e.g., IL-12 or IL-23 mRNA/protein/polypeptide/antibody). In some embodiments, the mRNA molecule(s) are injected into the subject directly. In some embodiments, one or more or all of the mRNA molecules are delivered as DNA which are then transcribed into mRNA in vivo.

In some embodiments, the OX40L is a human protein. In some embodiments, the OX40L is a full-length OX40L protein rather than a fragment or domain thereof, such as a soluble portion. In some embodiments, the OX40L is a full-length OX40L protein with different isoforms rather than a fragment or domain thereof, such as a soluble portion.

In some embodiments, the OX40 agonist is a polypeptide that includes at least an extracellular domain of full-length OX40L, which may be fused to a transmembrane domain and optionally an intracellular fragment of another protein.

In some embodiments, the OX40 agonist is a polypeptide that includes the extracellular domain, either alone or fused with a linker fragment (e.g., oligomerization domain) that can promote the formation of its homo-dimers, homo-trimers or homo-oligomers. Protein domains as such as the Fc fragment of immunoglobulins are commonly used to promote formation of homo-dimers.

In some embodiments, the oligomerization domain is capable of formation of homo-trimers (thus a “trimerization domain”). Trimerization domains are known in the art, such as the domains in trimeric proteins responsible for mediating association of the trimeric protein.

Example trimerization domains include the T4 bacteriophage fibritin trimerization motif (T4F), the GCN4 trimeric leucine zipper motif (GCN4), and the human collagen XVIII derived homotrimerization domain (TIE). In some embodiments, the trimerization domain is not longer than 100 amino acids, or not longer than 90, 80, 70, 60, or 50 amino acids.

In some embodiments, the fusion protein further includes a peptide linker between the OX40L extracellular domain and the trimerization domain. In some embodiments, the peptide linker is flexible.

In some embodiments, the distance between an OX40L extracellular domain and the trimerization domain is not longer than 100 amino acids, or not longer than 90, 80, 70, 60, 50, 40, 30, 25, 20, 15 or 10 amino acids. In some embodiments, the peptide linker is from 5 to 50 amino acid residues in length, preferably from 5 to 20 amino acid residues in length.

In some embodiment, the OX40 agonist is an agonist anti-OX40 antibody or an antigen-binding fragment thereof.

In some embodiments, the OX40 agonist mRNA includes the RNA sequence corresponding to the coding sequence of NM_003326 (SEQ ID NO:3). In some embodiments, the OX40L mRNA includes the RNA sequence corresponding to the coding sequence of NM_001297562 (SEQ ID NO:4). In some embodiment, the OX40 agonist mRNA encodes the protein sequence of NP_003317 (SEQ ID NO:1). In some embodiments, the OX40L mRNA encodes the protein sequence of NP_001284491 (SEQ ID NO:2).

In some embodiment, the OX40 agonist mRNA encodes a protein sequence that has at least 70%, 75%, 80%, 85%, 90%, 95%, 98%, or 99% sequence identity to SEQ ID NO:1 or residues 52-183 of SEQ ID NO:1. In some embodiments, the OX40 agonist mRNA encodes a protein sequence that has at least 70%, 75%, 80%, 85%, 90%, 95%, 98%, or 99% sequence identity to SEQ ID NO:2 or residues 2-133 of SEQ ID NO:2. In some embodiments, the protein sequence retains the activity of human OX40L or activates OX40.

In some embodiments, the OX40 agonist mRNA encodes the extracellular domain of NP_003317 (i.e., residues 52-183 of SEQ ID NO:1, or a peptide having at least 70%, 75%, 80%, 85%, 90%, 95%, 98%, or 99% sequence identity to residues 52-183 of SEQ ID NO:1). In some embodiments, the OX40 agonist mRNA encodes the extracellular domain of NP_001284491 (i.e., residues 2-133 of SEQ ID NO:2, or a peptide having at least 70%, 75%, 80%, 85%, 90%, 95%, 98%, or 99% sequence identity to residues 2-133 of SEQ ID NO:2).

In some embodiments, the extracellular domain of OX40L can be fused to the transmembrane domain and optionally an intracellular fragment of another protein, such that the fusion protein can be anchored to the plasma membrane. The transmembrane domain and intracellular fragment can be from any protein, such as a human protein, in particularly those that are expressed on the membranes of cells in a tissue where OX40L is desired to be expressed.

A transmembrane domain may be derived either from any membrane-bound or transmembrane protein, such as an alpha, beta or zeta chain of a T-cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CD5, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, the IL-12 is human IL-12. In some embodiments, the IL-12 includes IL-12A (p35). In some embodiments, the IL-12 includes IL-12B (p40). In some embodiment, the IL-12 mRNA includes a mRNA encoding IL-12A and a mRNA encoding IL-12B. In some embodiment, the IL-12 mRNA includes a mRNA encoding both IL-12A and IL-12B.

In some embodiments, the IL-12A mRNA includes the mRNA sequence corresponding to the coding sequence of NM_000882 (SEQ ID NO:9). In some embodiments, the IL-12A mRNA encodes the protein sequence of NP_000873 (SEQ ID NO:5), or the mature protein (residues 57-253 of SEQ ID NO:5).

In some embodiments, the IL-12A mRNA includes the mRNA sequence corresponding to the coding sequence of NM_001354582 (SEQ ID NO:10). In some embodiments, the IL-12A mRNA encodes the protein sequence of NP_001341511 (SEQ ID NO: 6), or the mature protein (residues 57-239 of SEQ ID NO:6).

In some embodiments, the IL-12A mRNA includes the mRNA sequence corresponding to the coding sequence of NM_001354583 (SEQ ID NO:11). In some embodiments, the IL-12A mRNA encodes the protein sequence of NP_001341512 (SEQ ID NO: 7), or the mature protein (residues 57-215 of SEQ ID NO:7).

In some embodiments, the IL-12A mRNA includes the mRNA sequence corresponding to the coding sequence of NM_001397992 (SEQ ID NO:12). In some embodiments, the IL-12A mRNA encodes the protein sequence of NP_001384921 (SEQ ID NO: 8), or the mature protein (residues 23-219 of SEQ ID NO:8).

In some embodiments, the IL-12B mRNA includes the mRNA sequence corresponding to the coding sequence of NM_002187 (SEQ ID NO:14). In some embodiments, the IL-12A mRNA encodes the protein sequence of NP_002178 (SEQ ID NO:13), or the mature protein (residues 23-328 of SEQ ID NO:13).

TABLE A
Sequences

| | | SEQ ID |
|---|---|---|
| Name | Sequence | NO: |
| OX40L Isoform 1 | MERVQPLEENVGNAARPRFERNKLLLVASVIQGLGLLLCFTYICLHFSAL | 1 |
| protein | QVSHRYPRIQSIKVQFTEYKKEKGFILTSQKEDEIMKVQNNSVIINCDGF | |
| (NP_003317) | YLISLKGYFSQEVNISLHYQKDEEPLFQLKKVRSVNSLMVASLTYKDKVY | |
| (extracellular: | LNVTTDNTSLDDFHVNGGELILIHQNPGEFCVL | |
| 52-183 | | |
| (underlined)) | | |
| OX40L Isoform 2 | MVSHRYPRIQSIKVQFTEYKKEKGFILTSQKEDEIMKVQNNSVIINCDGF | 2 |
| protein | YLISLKGYFSQEVNISLHYQKDEEPLFQLKKVRSVNSLMVASLTYKDKVY | |
| (NP_001284491) | LNVTTDNTSLDDFHVNGGELILIHQNPGEFCVL | |
| OX40L Isoform 1 | auggaaaggguccaaccccuggaagagaaugugggaaaugcagccaggcc | 3 |
| mRNA | aagauucgagaggaacaagcuauugcugguggccucuguaauucagggac | |
| (NM_003326) | uggggcugcuccugugcuucaccuacaucugccugcacuucucugcucuu | |
| coding portion | cagguaucacaucgguauccucgaauucaaaguaucaaaguacaauuuac | |
| | cgaauauaagaaggagaaagguuucauccucacuucccaaaaggaggaug | |
| | aaaucaugaaggugcagaacaacucagucaucaucaacugugauggguuu | |
| | uaucucaucucccugaagggcuacuucucccaggaagucaacauuagccu | |
| | ucauuaccagaaggaugaggagccccucuuccaacugaagaaggucaggu | |
| | cugucaacuccuugaugguggccucucugacuuacaaagacaaagucuac | |
| | uugaaugugaccacugacaauaccucccuggaugacuuccaugugaaugg | |
| | cggagaacugauucuuauccaucaaaauccuggugaauucuguguccuuu | |
| | ga | |
| OX40L Isoform 2 | augguaucacaucgguauccucgaauucaaaguaucaaaguacaauuuac | 4 |
| mRNA | cgaauauaagaaggagaaagguuucauccucacuucccaaaaggaggaug | |
| (NM_001297562) | aaaucaugaaggugcagaacaacucagucaucaucaacugugauggguuu | |
| coding portion | uaucucaucucccugaagggcuacuucucccaggaagucaacauuagccu | |
| | ucauuaccagaaggaugaggagccccucuuccaacugaagaaggucaggu | |
| | cugucaacuccuugaugguggccucucugacuuacaaagacaaagucuac | |
| | uugaaugugaccacugacaauaccucccuggaugacuuccaugugaaugg | |
| | cggagaacugauucuuauccaucaaaauccuggugaauucuguguccuuu | |
| | ga | |
| IL-12A isoform 1 | MWPPGSASQPPPSPAAATGLHPAARPVSLQCRLSMCPARSLLLVATLVLL | 5 |
| protein | DHLSLARNLPVATPDPGMFPCLHHSQNLLRAVSNMLQKARQTLEFYPCTS | |
| (NP_000873) | EEIDHEDITKDKTSTVEACLPLELTKNESCLNSRETSFITNGSCLASRKT | |
| (mature protein: | SFMMALCLSSIYEDLKMYQVEFKTMNAKLLMDPKRQIFLDQNMLAVIDEL | |
| 57-253 | MQALNFNSETVPQKSSLEEPDFYKTKIKLCILLHAFRIRAVTIDRVMSYL | |
| (underlined)) | NAS | |
| IL-12A isoform 2 | MWPPGSASQPPPSPAAATGLHPAARPVSLQCRLSMCPARSLLLVATLVLL | 6 |
| protein | DHLSLARNLPVATPDPGMFPCLHHSQNLLRAVSNMLQKARQTLEFYPCTS | |
| (NP_001341511) | EEIDHEDITKDKTSTVEACLPLELTKNGSCLASRKTSFMMALCLSSIYED | |
| (mature protein: | LKMYQVEFKTMNAKLLMDPKRQIFLDQNMLAVIDELMQALNENSETVPQK | |
| 57-239 | SSLEEPDFYKTKIKLCILLHAFRIRAVTIDRVMSYLNAS | |
| (underlined)) | | |
| IL-12A isoform 3 | MWPPGSASQPPPSPAAATGLHPAARPVSLQCRLSMCPARSLLLVATLVLL | 7 |
| protein | DHLSLARNLPVATPDPGMFPCLHHSQNLLRAVSNMLQKNESCLNSRETSF | |
| (NP_001341512) | ITNGSCLASRKTSFMMALCLSSIYEDLKMYQVEFKTMNAKLLMDPKRQIF | |
| (mature protein: | LDQNMLAVIDELMQALNENSETVPQKSSLEEPDFYKTKIKLCILLHAFRI | |
| 57-215 | RAVTIDRVMSYLNAS | |
| (underlined)) | | |
| IL-12A isoform 4 | MCPARSLLLVATLVLLDHLSLARNLPVATPDPGMFPCLHHSQNLLRAVSN | 8 |
| protein | MLQKARQTLEFYPCTSEEIDHEDITKDKTSTVEACLPLELTKNESCLNSR | |
| (NP_001384921) | ETSFITNGSCLASRKTSFMMALCLSSIYEDLKMYQVEFKTMNAKLLMDPK | |
| (mature protein: | RQTFLDQNMLAVIDELMQALNENSETVPQKSSLEEPDFYKTKIKLCILLH | |
| 23-219 | AFRIRAVTIDRVMSYLNAS | |
| (underlined)) | | |
| IL-12A isoform 1 | auguggcccccugggucagccucccagccaccgcccucaccugccgcggc | 9 |
| mRNA | cacaggucugcauccagcggcucgcccugugucccugcagugccggcuca | |
| (NM_000882) | gcauguguccagcgcgcagccuccuccuuguggcuacccugguccuccug | |
| coding portion | gaccaccucaguuuggccagaaaccuccccguggccacuccagacccagg | |
| | aauguucccaugccuucaccacucccaaaaccugcugagggccgucagca | |
| | acaugcuccagaaggccagacaaacucuagaauuuuacccuugcacuucu | |
| | gaagagauugaucaugaagauaucacaaaagauaaaaccagcacagugga | |
| | ggccuguuuaccauuggaauuaaccaagaaugagaguugccuaaauucca | |
| | gagagaccucuuucauaacuaaugggaguugccuggccuccagaaagacc | |
| | ucuuuuaugauggcccugugccuuaguaguauuuaugaagacuugaagau | |
| | guaccagguggaguucaagaccaugaaugcaaagcuucugauggauccua | |
| | agaggcagaucuuucuagaucaaaacaugcuggcaguuauugaugagcug | |
| | augcaggcccugaauuucaacagugagacugugccacaaaaauccucccu | |
| | ugaagaaccggauuuuuauaaaacuaaaaucaagcucugcauacuucuuc | |
| | augcuuucagaauucgggcagugacuauugauagagugaugagcuaucug | |
| | aaugcuuccuaa | |
| IL-12A isoform 2 | auguggcccccugggucagccucccagccaccgcccucaccugccgcggc | 10 |
| mRNA | cacaggucugcauccagcggcucgcccugugucccugcagugccggcuca | |
| (NM_001354582) | gcauguguccagcgcgcagccuccuccuuguggcuacccugguccuccug | |
| coding portion | gaccaccucaguuuggccagaaaccuccccguggccacuccagacccagg | |
| | aauguucccaugccuucaccacucccaaaaccugcugagggccgucagca | |
| | acaugcuccagaaggccagacaaacucuagaauuuuacccuugcacuucu | |
| | gaagagauugaucaugaagauaucacaaaagauaaaaccagcacagugga | |
| | ggccuguuuaccauuggaauuaaccaagaaugggaguugccuggccucca | |
| | gaaagaccucuuuuaugauggcccugugccuuaguaguauuuaugaagac | |
| | uugaagauguaccagguggaguucaagaccaugaaugcaaagcuucugau | |
| | ggauccuaagaggcagaucuuucuagaucaaaacaugcuggcaguuauug | |
| | augagcugaugcaggcccugaauuucaacagugagacugugccacaaaaa | |
| | uccucccuugaagaaccggauuuuuauaaaacuaaaaucaagcucugcau | |
| | acuucuucaugcuuucagaauucgggcagugacuauugauagagugauga | |
| | gcuaucugaaugcuuccuaa | |
| IL-12A isoform 3 | auguggcccccugggucagccucccagccaccgcccucaccugccgcggc | 11 |
| mRNA | cacaggucugcauccagcggcucgcccugugucccugcagugccggcuca | |
| (NM_001354583) | gcauguguccagcgcgcagccuccuccuuguggcuacccugguccuccug | |
| coding portion | gaccaccucaguuuggccagaaaccuccccguggccacuccagacccagg | |
| | aauguucccaugccuucaccacucccaaaaccugcugagggccgucagca | |
| | acaugcuccagaagaaugagaguugccuaaauuccagagagaccucuuuc | |
| | auaacuaaugggaguugccuggccuccagaaagaccucuuuuaugauggc | |
| | ccugugccuuaguaguauuuaugaagacuugaagauguaccagguggagu | |
| | ucaagaccaugaaugcaaagcuucugauggauccuaagaggcagaucuuu | |
| | cuagaucaaaacaugcuggcaguuauugaugagcugaugcaggcccugaa | |
| | uuucaacagugagacugugccacaaaaauccucccuugaagaaccggauu | |
| | uuuauaaaacuaaaaucaagcucugcauacuucuucaugcuuucagaauu | |
| | cgggcagugacuauugauagagugaugagcuaucugaaugcuuccuaa | |
| IL-12A isoform 4 | auguguccagcgcgcagccuccuccuuguggcuacccugguccuccugga | 12 |
| mRNA | ccaccucaguuuggccagaaaccuccccguggccacuccagacccaggaa | |
| (NM_001397992) | uguucccaugccuucaccacucccaaaaccugcugagggccgucagcaac | |
| coding portion | augcuccagaaggccagacaaacucuagaauuuuacccuugcacuucuga | |
| | agagauugaucaugaagauaucacaaaagauaaaaccagcacaguggagg | |
| | ccuguuuaccauuggaauuaaccaagaaugagaguugccuaaauuccaga | |
| | gagaccucuuucauaacuaaugggaguugccuggccuccagaaagaccuc | |
| | uuuuaugauggcccugugccuuaguaguauuuaugaagacuugaagaugu | |
| | accagguggaguucaagaccaugaaugcaaagcuucugauggauccuaag | |
| | aggcagaucuuucuagaucaaaacaugcuggcaguuauugaugagcugau | |
| | gcaggcccugaauuucaacagugagacugugccacaaaaauccucccuug | |
| | aagaaccggauuuuuauaaaacuaaaaucaagcucugcauacuucuucau | |
| | gcuuucagaauucgggcagugacuauugauagagugaugagcuaucugaa | |
| | ugcuuccuaa | |
| IL-12B protein | MCHQQLVISWFSLVFLASPLVAIWELKKDVYVVELDWYPDAPGEMVVLTC | 13 |
| (NP_002178) | DTPEEDGITWTLDQSSEVLGSGKTLTIQVKEFGDAGQYTCHKGGEVLSHS | |
| (mature protein: | LLLLHKKEDGIWSTDILKDQKEPKNKTFLRCEAKNYSGRFTCWWLTTIST | |
| 23-328 | DLTFSVKSSRGSSDPQGVTCGAATLSAERVRGDNKEYEYSVECQEDSACP | |
| (underlined)) | AAEESLPIEVMVDAVHKLKYENYTSSFFIRDIIKPDPPKNLQLKPLKNSR | |
| | QVEVSWEYPDTWSTPHSYFSLTFCVQVQGKSKREKKDRVFTDKTSATVIC | |
| | RKNASISVRAQDRYYSSSWSEWASVPCS | |
| IL-12B mRNA | aucuccccucguggccauaugggaacugaagaaagauguuuaugucguag | 14 |
| (NM_002187) | aauuggauugguauccggaugccccuggagaaauggugguccucaccugu | |
| coding portion | gacaccccugaagaagaugguaucaccuggaccuuggaccagagcaguga | |
| | ggucuuaggcucuggcaaaacccugaccauccaagucaaagaguuuggag | |
| | augcuggccaguacaccugucacaaaggaggcgagguucuaagccauucg | |
| | cuccugcugcuucacaaaaaggaagauggaauuugguccacugauauuuu | |
| | aaaggaccagaaagaacccaaaaauaagaccuuucuaagaugcgaggcca | |
| | agaauuauucuggacguuucaccugcugguggcugacgacaaucaguacu | |
| | gauuugacauucagugucaaaagcagcagaggcucuucugacccccaagg | |
| | ggugacgugcggagcugcuacacucucugcagagagagucagaggggaca | |
| | acaaggaguaugaguacucaguggagugccaggaggacagugccugccca | |
| | gcugcugaggagagucugcccauugaggucaugguggaugccguucacaa | |
| | gcucaaguaugaaaacuacaccagcagcuucuucaucagggacaucauca | |
| | aaccugacccacccaagaacuugcagcugaagccauuaaagaauucucgg | |
| | cagguggaggucagcugggaguacccugacaccuggaguacuccacauuc | |
| | cuacuucucccugacauucugcguucagguccagggcaagagcaagagag | |
| | aaaagaaagauagagucuucacggacaagaccucagccacggucaucugc | |
| | cgcaaaaaugccagcauuagcgugcgggcccaggaccgcuacuauagcuc | |
| | aucuuggagcgaaugggcaucugugcccugcaguua | |
| FADD protein | MDPFLVLLHSVSSSLSSSELTELKFLCLGRVGKRKLERVQSGLDLFSMLL | 15 |
| (NP_003815) | EQNDLEPGHTELLRELLASLRRHDLLRRVDDFEAGAAAGAAPGEEDLCAA | |
| | FNVICDNVGKDWRRLARQLKVSDTKIDSIEDRYPRNLTERVRESLRIWKN | |
| | TEKENATVAHLVGALRSCQMNLVADLVQEVQQARDLQNRSGAMSPMSWNS | |
| | DASTSEAS | |
| FADD mRNA | auggacccguuccuggugcugcugcacucggugucguccagccugucgag | 16 |
| (NM_003824) | cagcgagcugaccgagcucaaguuccuaugccucgggcgcgugggcaagc | |
| coding portion | gcaagcuggagcgcgugcagageggccuagaccucuucuccaugcugcug | |
| | gagcagaacgaccuggagcccgggcacaccgagcuccugcgcgagcugcu | |
| | cgccucccugcggcgccacgaccugcugcggcgcgucgacgacuucgagg | |
| | cgggggcggcggccggggccgcgccuggggaagaagaccugugugcagca | |
| | uuuaacgucauaugugauaauguggggaaagauuggagaaggcuggcucg | |
| | ucagcucaaagucucagacaccaagaucgacagcaucgaggacagauacc | |
| | cccgcaaccugacagagcgugugcgggagucacugagaaucuggaagaac | |
| | acagagaaggagaacgcaacaguggcccaccuggugggggcucucagguc | |
| | cugccagaugaaccugguggcugaccugguacaagagguucagcaggccc | |
| | gugaccuccagaacaggaguggggccauguccccgaugucauggaacuca | |
| | gacgcaucuaccuccgaagcguccuga | |
| MLKL protein | MENLKHIITLGQVIHKRCEEMKYCKKQCRRLGHRVLGLIKPLEMLQDQGK | 17 |
| (NP_689862) | RSVPSEKLTTAMNRFKAALEEANGEIEKFSNRSNICRFLTASQDKILFKD | |
| (four-helix | VNRKLSDVWKELSLLLQVEQRMPVSPISQGASWAQEDQQDADEDRRAFQM | |
| bundle | LRRDNEKIEASLRRLEINMKEIKETLRQYLPPKCMQEIPQEQIKEIKKEQ | |
| domain: 1-125 | LSGSPWILLRENEVSTLYKGEYHRAPVAIKVFKKLQAGSIAIVRQTENKE | |
| (underlined)) | IKTMKKFESPNILRIFGICIDETVTPPQFSIVMEYCELGTLRELLDREKD | |
| | LTLGKRMVLVLGAARGLYRLHHSEAPELHGKIRSSNFLVTQGYQVKLAGF | |
| | ELRKTQTSMSLGTTREKTDRVKSTAYLSPQELEDVFYQYDVKSEIYSFGI | |
| | VLWEIATGDIPFQGCNSEKIRKLVAVKRQQEPLGEDCPSELREIIDECRA | |
| | HDPSVRPSVDEILKKLSTFSK | |
| MLKL mRNA | auggaaaauuugaagcauauuaucacccuuggccaggucauccacaaacg | 18 |
| (NM_152649) | gugugaagagaugaaauacugcaagaaacagugccggcgccugggccacc | |
| coding portion | gcguccucggccugaucaagccucuggagaugcuccaggaccaaggaaag | |
| | aggagcgugcccucugagaaguuaaccacagccaugaaccgcuucaaggc | |
| | ugcccuggaggaggcuaauggggagauagaaaaguucagcaauagaucca | |
| | auaucugcagguuucuaacagcaagccaggacaaaauacucuucaaggac | |
| | gugaacaggaagcugagugaugucuggaaggagcucucgcuguuacuuca | |
| | gguugagcaacgcaugccuguuucacccauaagccaaggagcguccuggg | |
| | cacaggaagaucagcaggaugcagacgaagacaggcgagcuuuccagaug | |
| | cuaagaagagauaaugaaaaaauagaagcuucacugagacgauuagaaau | |
| | caacaugaaagaaaucaaggaaacuuugaggcaguauuuaccaccaaaau | |
| | gcaugcaggagaucccgcaagagcaaaucaaggagaucaagaaggagcag | |
| | cuuucaggauccccguggauucugcuaagggaaaaugaagucagcacacu | |
| | uuauaaaggagaauaccacagagcuccaguggccauaaaaguauucaaaa | |
| | aacuccaggcuggcagcauugcaauagugaggcagacuuucaauaaggag | |
| | aucaaaaccaugaagaaauucgaaucucccaacauccugcguauauuugg | |
| | gauuugcauugaugaaacagugacuccgccucaauucuccauugucaugg | |
| | aguacugugaacucgggacccugagggagcuguuggauagggaaaaagac | |
| | cucacacuuggcaagcgcaugguccuaguccugggggcagcccgaggccu | |
| | auaccggcuacaccauucagaagcaccugaacuccacggaaaaaucagaa | |
| | gcucaaacuuccugguaacucaaggcuaccaagugaagcuugcaggauuu | |
| | gaguugaggaaaacacagacuuccaugaguuugggaacuacgagagaaaa | |
| | gacagacagagucaaaucuacagcauaucucucaccucaggaacuggaag | |
| | auguauuuuaucaauaugauguaaagucugaaauauacagcuuuggaauc | |
| | guccucugggaaaucgccacuggagauaucccguuucaaggcuguaauuc | |
| | ugagaagauccgcaagcugguggcugugaagcggcagcaggagccacugg | |
| | gugaagacugcccuucagagcugcgggagaucauugaugagugccgggcc | |
| | caugaucccucugugcggcccucuguggaugaaaucuuaaagaaacucuc | |
| | caccuuuucuaaguag | |
| Group H 5′ UTR | gaauaaacuaguauucuucugguccccacagacucagagagaacccgcca | 19 |
| | cc | |
| Group H 3′ UTR | cggguggcaucccugugaccccuccccagugccucuccuggcccuggaag | 20 |
| | uugccacuccagugcccaccagccuuguccuaauaaaauuaaguugcauc | |
| | aagcu | |
| IL-12 Seq H | augugucaccagcagcuggucaucagcugguucagccugguguuccuggc | 21 |
| | cucuccucugguggccaucugggagcugaagaaagacguguacguggugg | |
| | aacuggacugguaucccgaugcuccuggcgagaugguggugcugaccugc | |
| | gauaccccugaagaggacggcaucaccuggacacuggaucagucuagcga | |
| | ggugcucggcagcggcaagacccugaccauccaagugaaagaguuuggcg | |
| | acgccggccaguacaccugucacaaaggcggagaagugcugagccacagc | |
| | cugcugcugcuccacaagaaagaggauggcauuuggagcaccgacauccu | |
| | gaaggaccagaaagagcccaagaacaagaccuuccugagaugcgaggcca | |
| | agaacuacagcggccgguucacauguugguggcugaccaccaucagcacc | |
| | gaccugaccuucagcgugaaguccagcagaggcagcagugauccucaggg | |
| | cguuacauguggcgccgcuacacugucugccgaaagagugcggggcgaca | |
| | acaaagaauacgaguacagcguggaaugccaagaggacagcgccugucca | |
| | gccgccgaagagucucugccuaucgaagugaugguggacgccgugcacaa | |
| | gcugaaguacgagaacuacaccuccagcuuuuucauccgggacaucauca | |
| | agcccgauccuccaaagaaccugcagcugaagccucugaagaacagcaga | |
| | cagguggaaguguccugggaguaccccgacaccuggucuacaccccacag | |
| | cuacuucagccugaccuuuugcgugcaagugcagggcaaguccaagcgcg | |
| | agaaaaaggaccggguguucaccgacaagaccagcgccaccgugaucugc | |
| | agaaagaacgccagcaucagcgucagagcccaggaccgguacuacagcag | |
| | cucuuggagcgaaugggccagcgugccauguucugguggaucuggcggag | |
| | gaagcggaggcggagguucuggcggugguggaagcgguggcggcucuggu | |
| | ggaagaaaucugccuguggccacuccugauccuggcauguucccuugucu | |
| | gcaccacucucagaaccugcugagagccguguccaacaugcugcagaagg | |
| | ccagacagacccuggaauucuaccccugcaccagcgaggaaaucgaccac | |
| | gaggacaucaccaaggauaagaccagcaccguggaagccugccugccucu | |
| | ggaacugaccaagaacgagagcugccugaacagccgggaaaccagcuuca | |
| | ucaccaacggcucuugccuggccagcagaaagaccuccuucaugauggcc | |
| | cugugccugagcagcaucuacgaggaccugaagauguaccagguggaauu | |
| | caagaccaugaacgccaagcugcugauggaccccaagcggcagaucuucc | |
| | uggaccagaauaugcuggccgugaucgacgagcugaugcaggcccugaac | |
| | uucaacagcgagacagugccccagaagucuagccuggaagaacccgacuu | |
| | cuacaagaccaagaucaagcugugcauccugcugcacgccuuccggauca | |
| | gagccgugaccaucgacagagugaugagcuaccugaacgccuccuaa | |
| IL-12 Seq #1 | augugucaccagcagcuggugaucagcugguucagcuugguguucuuggc | 22 |
| | cagcccacugguggccaucugggagcugaagaaggacguguacgucguug | |
| | agcuggacugguaccccgacgcuccaggggagaugguggugcugaccugc | |
| | gacaccccggaggaggacggcaucaccuggacccuggaccaguccagcga | |
| | agugcugggcagcgggaagaccuugaccauccaggucaaggaguucggcg | |
| | acgcuggccaguacaccugccacaaaggcggggaggugcugagccacagc | |
| | cugcugcuguugcacaagaaggaggacggcaucugguccacagacauucu | |
| | gaaggaccagaaggagccgaagaacaagaccuuccugcggugugaggcca | |
| | agaacuacagcggcagguucaccugcugguggcugaccaccaucagcacu | |
| | gaccugaccuucagcgucaagagcuccaggggcagcagcgacccccaggg | |
| | ugucacuuguggcgccgccacccucucagccgagagggugcgcggcgaca | |
| | acaaggaguacgaguacuccguggagugucaggaggacuccgccugcccc | |
| | gccgccgaggagucccugccgaucgaggucaugguggacgccguccacaa | |
| | gcugaaguaugagaauuauacuuccagcuucuucauucgggacaucauca | |
| | agcccgacccucccaagaaccugcagcugaagccccugaagaacagcagg | |
| | cagguggaggugagcugggaguacccggacaccuggagcaccccccauuc | |
| | uuacuucagcuugaccuuuugcgugcaggugcagggcaagagcaagcggg | |
| | agaagaaggacaggguguucacagacaagacgagcgccacggugaucugu | |
| | cgcaagaaugcgucgaucagcgugegcgcucaggacagguacuacagcuc | |
| | aagcugguccgagugggccagcguccccugcagcggcggcucagggggcg | |
| | gcuccggcggcgggggcuccggcggcggcggcagcggaggggguagcggc | |
| | gggaggaaccuucccgucgcuacccccgaccccggcauguuuccuugccu | |
| | gcaccacagccagaaccugcugcgggcgguaaguaacaugcugcagaagg | |
| | ccaggcagacccuggaauucuaccccugcaccuccgaggagaucgaccac | |
| | gaggauaucaccaaagauaagaccuccaccguggaggcgugccugccccu | |
| | ggagcucaccaagaacgagucuugccucaacagcagggaaaccuccuuca | |
| | ucaccaacggcagcugcuuggccagcaggaagaccagcuucaugauggcc | |
| | cuuugucugagcuccaucuacgaggaucugaagauguaucagguggaguu | |
| | caagacaaugaacgccaagcuguugauggaccccaagaggcagaucuucu | |
| | uggaucagaauaugcuggccguuaucgaugagcugaugcaggcccugaac | |
| | uucaauucugaaaccgugccgcagaaguccagcuuagaggagcccgacuu | |
| | cuacaagaccaagauuaagcugugcauucugcugcacgcuuucagaauca | |
| | gggccgucaccaucgauagggucaugucauaucugaacgccagcuga | |
| IL-12 Seq #2 | augugccaccagcagcuggugaucagcugguucucgcuggucuuccucgc | 23 |
| | guccccucugguagcgaucugggagcucaagaaggaugucuacguggucg | |
| | agcuggacugguacccugacgccccgggggagaugguggucuugaccugc | |
| | gacaccccagaggaggacggcaucaccuggacucuugaccagagcucaga | |
| | gguucugggcucuggcaagaccuugaccauccaggucaaggaguucgggg | |
| | augccggccaguacaccugccacaagggcggggaggugcugagccacucc | |
| | cuccuccuucugcacaagaaggaggacgggaucugguccacagacauucu | |
| | gaaggaccagaaggagcccaagaacaagaccuuccugaggugcgaggcca | |
| | agaacuacuccggcagguucaccugcuggugguugaccaccaucuccacc | |
| | gacuugaccuucucggucaagucgucuagggguagcagcgacccgcaggg | |
| | cgugaccugcggggcugcuacccuuagcgccgagcgggugcggggcgaca | |
| | acaaggaguacgaguacagcguagagugccaggaggacucugccuguccc | |
| | gccgccgaggagucccugccgaucgaggucaugguggacgccgugcacaa | |
| | gcugaaguaugagaauuauaccagcuccuucuucauccgcgacaucauca | |
| | agccugacccgcccaagaaccugcagcugaagccgcugaagaacagccgu | |
| | cagguugaagugagcugggaguacccagacaccuggaguacuccccacag | |
| | cuacuucagccugaccuucugcgugcagguucagggcaagucaaagcggg | |
| | agaagaaggacaggguguucaccgacaagaccucggccaccgugaucugc | |
| | aggaagaacgcuagcauuucugugegggcccaggaccgcuacuacaguag | |
| | uagcugguccgagugggccagcguucccugcagcgguggcagcggcgggg | |
| | gcuccggcggcggcgggaguggaggcgggggaagcggcggggggucgggc | |
| | gguaggaaccugccgguugcuacccccgaccccggcauguuccccugccu | |
| | ccaccacucgcagaaccuucugcgagcugucagcaacaugcugcagaagg | |
| | ccaggcagacccuggaauucuacccuugcaccagcgaggagaucgaccac | |
| | gaagacaucacuaaagacaagaccuccaccguggaggcuugucuuccucu | |
| | ugagcucaccaagaacgagagcugccugaacucgagggaaaccagcuuca | |
| | ucacuaacgggagcugucuggcguccaggaagacuagcuucaugauggcu | |
| | cugugccugagcuccaucuacgaggacuugaagauguaccagguggaguu | |
| | caagaccaugaacgcaaagcugcucauggaucccaagaggcagaucuucc | |
| | uggaccagaacaugcucgccguuauugaugagcucaugcaggcccugaac | |
| | uucaacuccgaaaccgugccgcagaaguccagcuuggaggagcccgacuu | |
| | cuacaagaccaagaucaagcugugcauucugcugcacgcguuucggauca | |
| | gggccgucaccaucgacagggugaugagcuaccugaacgccagcuga | |
| IL-12 Seq #3 | augugccaucagcaacugguuaucagcugguucagcuugguguuucuggc | 24 |
| | uagcccccugguggccaucugggagcugaaaaaggacguguacguggugg | |
| | agcuggauugguaccccgacgccccgggcgagaugguggugcuuaccugc | |
| | gauacacccgaagaggauggaaucacuuggacgcuggaccaguccagcga | |
| | ggugcuggguuccggcaagacccucaccauacaggugaaggaguuuggcg | |
| | acgccggccaguacaccugucacaaaggcggggaggugcugagucauagc | |
| | cugcugcuccuucacaagaaggaggacggcaucugguccacagacauucu | |
| | gaaggaccagaaggagcccaagaacaaaaccuuucugaggugugaagcca | |
| | aaaacuacuccggcagguucaccugcugguggcuuacaaccaucucuacc | |
| | gaucugacuuucucagugaaaagcucaagggggucaagcgacccgcaggg | |
| | cgugaccugcggcgcggccacccugagcgcugagagagucaggggugaca | |
| | acaaggaguacgaauacuccguugagugucaggaggacagcgccugcccg | |
| | gcagccgaggagagccugcccaucgaggucaugguggacgccgugcacaa | |
| | gcugaaguaugaaaauuauaccagcucuuucuucaucagggauauuauca | |
| | agccugauccacccaagaaccugcagcuuaagccacugaagaacaguagg | |
| | cagguugagguuucuugggaguacccugauacguggagcacuccccauag | |
| | cuacuucagccugaccuucugugugcaggugcaaggcaagucuaagaggg | |
| | agaagaaagauaggguguucaccgacaagaccucugccaccgugaucugc | |
| | aggaagaacgcuagcauuuccguucgggcucaggacagguacuacucuag | |
| | cagcuggagcgagugggccucagucccuugcucuggcggcagugguggag | |
| | gcagcgggggcggcgguuccggcggcggcggcagcggcgguggcagcggc | |
| | ggcaggaaccugccgguugccacccccgaucccgggauguuccccugccu | |
| | ccaucacagccagaaucuauuacgugcaguguccaacauguugcagaagg | |
| | ccaggcagacucuugaguucuauccuugcacgagcgaggagaucgaccac | |
| | gaggacaucaccaaggacaagacaagcacuguggaggcuugucugccucu | |
| | ggagcugaccaagaacgagagcugccugaacagcagggaaaccucuuuca | |
| | ucacuaacggcagcugccuggccucucgcaagaccagcuucaugauggca | |
| | cugugucugagcaguaucuaugaggaccuuaagauguaccagguggaguu | |
| | uaagaccaugaacgccaagcugcugauggaccccaagaggcagauuuucu | |
| | uggaucagaacaugcucgccgucaucgacgagcucaugcaggcgcugaau | |
| | uucaacagugagacagugccucagaaguccagucuggaggagccugauuu | |
| | uuacaagacaaagaucaagcugugcauucugcugcacgccuuccguauca | |
| | gggccgucaccaucgacagagugaugagcuaccugaacgccucuuga | |
| IL-12 Seq #4 | augugccaccagcagcuggugaucagcugguucagcuugguguuccuggc | 25 |
| | cagcccacugguggccaucugggagcugaagaaggaugucuacguggucg | |
| | agcuggacugguaccccgacgccccaggggagaugguggugcugaccugc | |
| | gacaccccggaggaggacggcaucaccuggacccuggaccaguccagcga | |
| | ggugcugggcagcgggaagacccucaccauccaggugaaggaguucggcg | |
| | acgccggccaguacacuugccacaaggguggcgaggugcugagccacucc | |
| | cugcugcugcuucacaagaaggaggacgguaucugguccacagacauucu | |
| | gaaggaccagaaggagcccaagaacaagaccuuccugaggugcgaggcca | |
| | agaacuacuccggcagguucaccugcugguggcugaccaccaucaguacc | |
| | gaucugaccuucucugugaaguccagcagggggagcagugacccccaggg | |
| | ggucaccugcggcgccgccacucugagcgccgagagggugaggggggaca | |
| | acaaggaguacgaguacuccguggagugccaggaggacuccgccuguccc | |
| | gccgccgaggagucccugccaaucgaggucaugguggacgccgugcacaa | |
| | gcugaaguacgagaacuacaccagcuccuucuucauccgcgacaucauca | |
| | agccagacccccccaagaaccugcagcugaagccucugaagaacagcagg | |
| | cagguugaggugagcugggaguacccagacaccuggaguacuccccacag | |
| | cuacuucagccugaccuucugcgugcagguucaggggaagucuaagcggg | |
| | agaagaaggacaggguguucaccgacaagaccucggccaccgucaucugc | |
| | aggaagaaugcguccaucuccgugagggcgcaggacagauacuacuccuc | |
| | cuccuggagcgagugggcaucuguccccugcaguggcgggagcggcgggg | |
| | gcuccggcggcggcgggucaggcggagguggcagcggcggggggagcggg | |
| | ggcaggaaccugcccgucgccacccccgacccuggcauguuccccugccu | |
| | gcaccacucccagaaucugcugcgggccgugagcaacaugcuccagaagg | |
| | cccggcagacucuggaguucuauccuugcaccagcgaggagaucgaccac | |
| | gaggacaucacuaaagacaagaccuccaccguggaggcuugucugcccuu | |
| | ggagcugaccaagaacgagucuugccugaauagccgggaaaccagcuuca | |
| | ucaccaacggaagcugccuggcuagcaggaagaccagcuucaugauggcc | |
| | cugugcuugagcuccaucuacgaggaccugaagauguaccagguggaguu | |
| | caagaccaugaacgccaagcugcugauggaccccaagcggcagaucuucc | |
| | uggaucagaacaugcucgccgugaucgaugagcugaugcaggcccugaac | |
| | uucaauucugaaaccgugccgcagaaguccagcuuggaggagcccgacuu | |
| | cuacaagaccaagaucaagcugugcauucugcugcacgcuuucagaauca | |
| | gggccgucaccaucgacagggugaugagcuaccugaacgccagcuga | |
| IL-12 Seq #6 | augugccaccagcagcuggugaucagcugguucagccugguguuccuggc | 26 |
| | gagcccucugguggcaaucugggaguuaaagaaggacguguacguggugg | |
| | agcuggacugguaccccgaugcccccggcgagauggugguacugaccugc | |
| | gacacgcccgaggaggacggcaucaccuggacccuggaucagagcagcga | |
| | ggugcuggguagcggcaagacccuuaccauucaggugaaggaguucggcg | |
| | acgccggccaguacacuugccacaaggguggcgaggugcugagccauucu | |
| | cugcugcuucuucacaagaaagaggacgguaucugguccaccgacauccu | |
| | gaaggaucagaaggagccuaagaacaagaccuuucugcggugcgaggcca | |
| | agaauuacucaggcagguucaccugcugguggcugacuaccaucagcacc | |
| | gaccugacuuucucugugaaguccagcaggggaagcagugacccccaggg | |
| | gguuaccugcggcgccgccacccuuagcgccgaaagggugaggggcgaua | |
| | acaaggaguacgaauacucuguggagugucaggaggacagcgccugucca | |
| | gcugcugaggagagcuugccuauugagguuaugguggacgccguccacaa | |
| | gcugaaguaugagaauuauaccagcucguucuucaucagggacauaauca | |
| | aaccugaucccccuaagaacuugcagcugaagccacuaaagaacagcagg | |
| | cagguugagguaagcugggaguacccagacaccuggaguacuccgcacag | |
| | cuacuucagcuugaccuucugugugcagguucagggcaagucuaagaggg | |
| | agaagaaggacaggguguucaccgacaagaccucagccaccgugaucugc | |
| | aggaagaacgcaagcaucagcgugagggcccaggacagguacuacucaag | |
| | cagcuggagcgagugggccagcguccccugcagcggcggcagcggcggcg | |
| | guagcggcggcggggguagcggcggcggcgggucuggcggcggcagcggc | |
| | ggcagaaaccugcccgucgcuacccccgaccccgguauguucccuugcuu | |
| | gcaccacucucagaaucugcuuagggccgugagcaauaugcugcagaagg | |
| | ccaggcagacucuggaguucuacccguguacgagcgaggagaucgaucac | |
| | gaggacaucacuaaggacaaaaccucuaccgucgaggccugccugccgcu | |
| | ggagcugaccaagaacgaaucuugccugaacuccagggaaaccagcuuca | |
| | ucacgaacggcagcugccuggccagcaggaagaccucuuucaugauggcc | |
| | cugugccugagcagcaucuaugaggaccuuaagauguaucagguggaguu | |
| | uaagaccaugaacgccaagcugcugauggaccccaaacgccagauauuuc | |
| | uggaucagaacaugcuggccgugauugaugagcugaugcaagcccuuaac | |
| | uuuaacuccgaaaccgugccccagaagaguucgcuagaggagcccgauuu | |
| | uuacaagaccaagaucaaacugugcauccugcugcacgcguuuaggauca | |
| | gggcugugacaaucgaccgggugaugagcuaccugaacgccagcuaa | |
| OX40L Seq H | auggaaagagugcagccccuggaagagaacgugggcaaugccgccagacc | 27 |
| | uagauucgagcggaacaagcugcugcugguggccucugugauccaaggac | |
| | ugggacugcugcugugcuucaccuacaucugccugcacuucagegcccuc | |
| | caggugucccacagauaccccagaauccagagcaucaaggugcaguucac | |
| | cgaguacaagaaagagaagggcuucauccugaccagccagaaagaggacg | |
| | agaucaugaaggugcagaacaacagegugaucaucaacugcgacggcuuc | |
| | uaccugaucagccugaagggcuacuucagccaagaagugaauaucagccu | |
| | gcacuaccagaaggacgaggaaccccuguuccagcugaagaaagugcgga | |
| | gcgugaacagccugaugguggccagccugaccuacaaggacaagguguac | |
| | cugaacgugaccaccgacaacaccagccuggacgacuuccaugugaaugg | |
| | cggcgagcugauccugauccaucagaacccuggcgaguucugcgugcugu | |
| | aa | |
| OX40L Seq #7 | auggagcggguucagccccuggaggagaauguagguaacgcagccaggcc | 28 |
| | cagguucgagcggaacaagcugcugcuuguagcuuccgugauucagggcc | |
| | ugggccugcugcuguguuuuaccuacauuugccuccauuucagcgcucug | |
| | caggugucacacagguacccaaggauccaguccaucaaagugcaguucac | |
| | ggaguacaagaaggagaagggcuucauccucaccucucagaaggaagaug | |
| | agaucaugaaggugcagaacaacuccgugaucaucaacugcgacggcuuc | |
| | uaccucaucucucugaagggcuacuuuagccaggagguuaacauuagccu | |
| | ccacuaucagaaggaugaggagccguuguuccagcugaagaaggugaggu | |
| | ccgugaacucuuugaugguggcauccuugaccuacaaggacaagguguac | |
| | cugaaugugaccaccgacaacacgagccuggaugacuuccaugugaaugg | |
| | aggggagcugauccugauccaucagaacccaggggaguucugcguccucu | |
| | ga | |
| OX40L Seq #8 | auggagagggugcagccccuggaggagaacguuggcaacgccgcgaggcc | 29 |
| | cagguucgagcgcaacaagcuccuccugguugccagcgugauccaggggc | |
| | uggggcugcuguugugcuuuacauacauuuguuugcacuucagcgcccug | |
| | caggugucacacagguacccucguauccagaguauuaaggugcaguucac | |
| | cgaguacaagaaggagaagggcuucaucuugaccucccagaaggaggacg | |
| | agauaaugaagguccagaacaacuccguaaucaucaacugugacggauuu | |
| | uaccuuaucagcuugaagggguacuucucccaggaggugaacaucagccu | |
| | ccacuaccagaaggacgaggagccccuuuuccagcugaagaaggugagau | |
| | ccgucaacagucugaugguugcgagucugaccuacaaggauaagguguac | |
| | cugaaugugaccaccgacaacaccucucucgaugacuuucacgugaacgg | |
| | gggcgagcugauucugauccaucagaaucccggcgaguucugcguucugu | |
| | ga | |
| OX40L Seq #9 | auggagagggugcagccccuggaggagaacgucggcaaugccgccaggcc | 30 |
| | uagguuugagaggaacaaacugcugcucguggccagcgugauccagggcc | |
| | ugggccugcuccucugcuucaccuauaucugccugcacuucagugcacug | |
| | caggugucacauagguacccuaggauucagaguaucaagguacaguucac | |
| | ugaguauaagaaggagaagggcuuuauccucacuucucagaaggaggaug | |
| | agaucaugaagguacagaacaacucggugaucauaaacugugacggguuu | |
| | uaccuuauuucacugaagggcuacuucucucaggaggugaacauuaguuu | |
| | acacuaccagaaagacgaggagcccuuauuucagcugaagaaggugaggu | |
| | ccguuaauagucuuauggucgccagucugaccuauaaggauaagguguac | |
| | cugaacgugacaacggacaauaccucccuugacgacuuccacguuaacgg | |
| | cggugaacugauuuugauccaccagaauccuggcgaguucugugugcucu | |
| | aa | |
| OX40L Seq #10 | auggagcggguccagccccuggaggagaacguuggcaacgcagegcggcc | 31 |
| | cagguucgagaggaacaagcugcugcugguugccagcgugauccaggggc | |
| | ugggccugcuccugugcuucaccuacaucugccugcacuucagcgcacug | |
| | caggugagccacagguaucccaggauacagagcaucaaggugcaguucac | |
| | cgaguacaagaaggagaagggcuucauccucacuucccagaaggaggaug | |
| | agaucaugaaggugcagaacaacagcgugaucaucaacugugacggcuuc | |
| | uaccugaucagccugaagggguacuucagccaggaggugaacaucagccu | |
| | ccacuaccagaaggaugaggagccccuguuucagcugaagaaggugaggu | |
| | ccgucaacagucugauggucgcguccuugaccuacaaggacaagguguac | |
| | cugaacgugaccaccgacaacaccagccuggacgacuuccaugugaacgg | |
| | cggugagcugaucuugauucaccagaacccuggugaguucuguguccugu | |
| | ga | |
| OX40L Seq #11 | auggagagggugcagccccuggaggagaacguggguaacgcggcccggcc | 32 |
| | ccgguucgagcgcaacaagcugcugcuuguugcguccgugauccaggggc | |
| | ugggccugcuccugugcuucaccuacaucugccugcacuucagcgcguug | |
| | caggugucgcacagguacccgeggauccagagcaucaaggugcaguucac | |
| | ggaguacaagaaggagaagggcuucauccucaccucccagaaggaggaug | |
| | agaucaugaaggugcagaacaacagegugaucaucaacugugacggcuuc | |
| | uaccucaucagcuugaagggguacuucucgcaggaggugaacaucagccu | |
| | ccacuaccagaaggacgaggagccccucuuccagcugaagaaggugaggu | |
| | ccgucaacagcuugauggucgcguccuugaccuacaaggacaagguguac | |
| | cucaacgugaccacggacaacacgucccuggacgacuuccacgugaacgg | |
| | gggugaguugauacucauccaccagaacccgggggaguucugcguccucu | |
| | ag | |
| OX40L Seq #12 | auggagcgcgugcagccgcuggaggaaaacgucgggaacgccgcucgccc | 33 |
| | uagguucgagaggaauaagcuucuccugguggcgagegugauccagggac | |
| | ugggccugcuccugugcuucaccuacaucugccugcacuucagcgcguug | |
| | caggugagccauagauacccuaggauccagaguauuaaggugcaguucac | |
| | cgaguacaagaaggagaagggauucauccucaccagucagaaggaggacg | |
| | agaucaugaagguucagaacaauuccgugauuaucaacugugauggcuuc | |
| | uaccucaucagccugaagggcuauuucagucaggaagugaacauuucccu | |
| | gcacuaucaaaaggaugaggagccccuuuuucagcugaagaaggugaggu | |
| | ccguuaacucucugaugguggccucucugaccuacaaggacaagguguac | |
| | cugaacgugaccaccgauaauacuucccuggaugauuuccacguuaacgg | |
| | cggcgagcugauccugauucaccagaaucccggcgaguuuugcgugcucu | |
| | ag | |

In some embodiments, the OX40L mRNA (or mRNA encoding a protein/polypeptide/antibody or their combination that activates OX40) and the IL-12 mRNA are separate mRNA molecules. In some embodiments, the OX40L (or mRNA encoding a protein/polypeptide/antibody or their combination that activates OX40) coding sequence and the IL-12 coding sequence are included in the same mRNA molecule which can be translated into a fusion protein/polypeptide or separate proteins/polypeptides.

In some embodiments, one of the mRNA encodes OX40L, such as human OX40L. In some embodiments, the encoded protein has the amino acid sequence of SEQ ID NO:1. In some embodiments, the encoded protein has an amino acid sequence that has at least 70%, 80%, 85%, 90%, 95%, 98%, or 99% sequence identity to SEQ ID NO:1. In some embodiments, a fragment of the encoded protein has an amino acid sequence that has at least 90%, 95%, 98%, or 99% sequence identity to a fragment in SEQ ID NO:1. In some embodiments, the encoded protein has the amino acid sequence of SEQ ID NO:2. In some embodiments, the encoded protein has an amino acid sequence that has at least 70%, 80%, 85%, 90%, 95%, 98%, or 99% sequence identity to SEQ ID NO:2. In some embodiments, a fragment of the encoded protein has an amino acid sequence that has at least 90%, 95%, 98%, or 99% sequence identity to a fragment in SEQ ID NO:2. (Someone may make a fusion protein which contains the extracellular domain of OX40L and the intracellular and transmembrane domains from other proteins).

In some embodiments, the mRNA includes the nucleic acid sequence of SEQ ID NO:3. In some embodiments, the mRNA includes the nucleic acid sequence that has at least 70%, 80%, 85%, 90%, 95%, 98%, or 99% sequence identity to SEQ ID NO:3. In some embodiments, the mRNA includes the nucleic acid sequence of SEQ ID NO:4. In some embodiments, the mRNA includes the nucleic acid sequence that has at least 70%, 80%, 85%, 90%, 95%, 98%, or 99% sequence identity to SEQ ID NO:4.

In some embodiments, one of the mRNA encodes IL-12A, such as human IL-12A. In some embodiments, the encoded protein has the amino acid sequence of SEQ ID NO:5. In some embodiments, the encoded protein has an amino acid sequence that has at least 70%, 80%, 85%, 90%, 95%, 98%, or 99% sequence identity to SEQ ID NO:5. In some embodiments, the encoded protein has the amino acid sequence of SEQ ID NO:6. In some embodiments, the encoded protein has an amino acid sequence that has at least 70%, 80%, 85%, 90%, 95%, 98%, or 99% sequence identity to SEQ ID NO:6. In some embodiments, the encoded protein has the amino acid sequence of SEQ ID NO:7. In some embodiments, the encoded protein has an amino acid sequence that has at least 70%, 80%, 85%, 90%, 95%, 98%, or 99% sequence identity to SEQ ID NO:7. In some embodiments, the encoded protein has the amino acid sequence of SEQ ID NO:8. In some embodiments, the encoded protein has an amino acid sequence that has at least 70%, 80%, 85%, 90%, 95%, 98%, or 99% sequence identity to SEQ ID NO: 8.

In some embodiments, the mRNA includes the nucleic acid sequence of SEQ ID NO:9. In some embodiments, the mRNA includes the nucleic acid sequence that has at least 70%, 80%, 85%, 90%, 95%, 98%, or 99% sequence identity to SEQ ID NO:9. In some embodiments, the mRNA includes the nucleic acid sequence of SEQ ID NO:10. In some embodiments, the mRNA includes the nucleic acid sequence that has at least 70%, 80%, 85%, 90%, 95%, 98%, or 99% sequence identity to SEQ ID NO:10. In some embodiments, the mRNA includes the nucleic acid sequence of SEQ ID NO:11. In some embodiments, the mRNA includes the nucleic acid sequence that has at least 70%, 80%, 85%, 90%, 95%, 98%, or 99% sequence identity to SEQ ID NO:11. In some embodiments, the mRNA includes the nucleic acid sequence of SEQ ID NO:12. In some embodiments, the mRNA includes the nucleic acid sequence that has at least 70%, 80%, 85%, 90%, 95%, 98%, or 99% sequence identity to SEQ ID NO: 12.

In some embodiments, one of the mRNA encodes IL-12B, such as human IL-12B. In some embodiments, the encoded protein has the amino acid sequence of SEQ ID NO:13. In some embodiments, the encoded protein has an amino acid sequence that has at least 70%, 80%, 85%, 90%, 95%, 98%, or 99% sequence identity to SEQ ID NO:13.

In some embodiments, the mRNA includes the nucleic acid sequence of SEQ ID NO: 14. In some embodiments, the mRNA includes the nucleic acid sequence that has at least 70%, 80%, 85%, 90%, 95%, 98%, or 99% sequence identity to SEQ ID NO: 14.

In some embodiments, the one or more mRNA encode, collectively OX40L (or a protein/polypeptide/antibody or their combination that activates OX40) and IL-12A. In some embodiments, the one or more mRNA encode, collectively OX40L (or a protein/polypeptide/antibody or their combination that activates OX40) and IL-12B. In some embodiments, the one or more mRNA encode, collectively OX40L (or a protein/polypeptide/antibody or their combination that activates OX40), IL-12A and IL-12B.

When the mRNA molecules encoding IL-12 and OX40L (or a soluble counterpart) are provided on separate molecules, their ratios can be adjusted as needed. In one embodiment, the mass ratio between the IL-12 mRNA and the OX40L mRNA is from 1:0.5 to 1:6, without limitation. In one embodiment, the mass ratio between the IL-12 mRNA and the OX40L mRNA is from 1:0.5 to 1:5. In one embodiment, the mass ratio between the IL-12 mRNA and the OX40L mRNA is from 1:0.5 to 1:4. In one embodiment, the mass ratio between the IL-12 mRNA and the OX40L mRNA is from 1:0.75 to 1:6. In one embodiment, the mass ratio between the IL-12 mRNA and the OX40L mRNA is from 1:0.75 to 1:5. In one embodiment, the mass ratio between the IL-12 mRNA and the OX40L mRNA is from 1:0.75 to 1:4. In one embodiment, the mass ratio between the IL-12 mRNA and the OX40L mRNA is from 1:0.8 to 1:5. In one embodiment, the mass ratio between the IL-12 mRNA and the OX40L mRNA is from 1:0.8 to 1:4. In one embodiment, the mass ratio between the IL-12 mRNA and the OX40L mRNA is from 1:0.8 to 1:3.

In one embodiment, the mass ratio between the IL-12 mRNA and the OX40L mRNA is from 1:0.9 to 1:5. In one embodiment, the mass ratio between the IL-12 mRNA and the OX40L mRNA is from 1:0.9 to 1:4. In one embodiment, the mass ratio between the IL-12 mRNA and the OX40L mRNA is from 1:0.9 to 1:3.5. In one embodiment, the mass ratio between the IL-12 mRNA and the OX40L mRNA is from 1:0.9 to 1:3. In one embodiment, the mass ratio between the IL-12 mRNA and the OX40L mRNA is from 1:0.9 to 1:2.5.

In one embodiment, the mass ratio between the IL-12 mRNA and the OX40L mRNA is from 1:0.9 to 1:2. In one embodiment, the mass ratio between the IL-12 mRNA and the OX40L mRNA is from 1:1 to 1:4. In one embodiment, the mass ratio between the IL-12 mRNA and the OX40L mRNA is from 1:1 to 1:3. In one embodiment, the mass ratio between the IL-12 mRNA and the OX40L mRNA is from 1:1 to 1:2.5. In one embodiment, the mass ratio between the IL-12 mRNA and the OX40L mRNA is from 1:1 to 1:2.

In some embodiments, the OX40L is a full-length OX40L protein. In some embodiments, the OX40L is Fc-OX40L (soluble fragment).

Codon-optimized coding sequences for IL-12 and OX40L are also provided, and have been tested. In some embodiments, the mRNA sequence for IL-12 is selected from SEQ ID NO: 21-26. In some embodiments, the mRNA sequence is selected from the group consisting of SEQ ID NO:27-33. In some embodiments, the mRNA sequence is selected from the group consisting of SEQ ID NO:27, 28, 30, 32 and 33.

As shown in the experimental examples, addition of other potential therapeutic agents may not increase the therapeutic efficacy of the instant combination of mRNA. Accordingly, in one embodiment, the treatment method or use or composition does not include some other types of mRNA.

In one embodiment, excluded are mRNAs encoding an immune checkpoint inhibitor such as PD-L1, PD-1 and CTLA-4. In one embodiment, excluded are mRNAs encoding an interferon, such as IFN-α, IFN-β, or IFN-γ. In one embodiment, excluded are mRNAs encoding another of the IL-12 family, such as IL-23, IL-27 and IL-35. In one embodiment, excluded are mRNAs encoding other cytokines, such as IL-18.

It is appreciated that one or more of these factors may still be included in certain scenarios. In some embodiments, one or more of these, or one or more others may be included. For instance, in one embodiment, the method, use or composition further includes mRNA encoding part or full length of an immunomodulatory factor, such as FADD, MLKL, CD27, CD28, CD40, CD122, CD137, GITR, GSDMD, A2AR, CD276, VTCN1, BTLA, CTLA-4, IDO, LAG3, KIR, NOX2, PD-1, TIM-3, VISTA, SIGLEC7, SIGLEC9, IL-2, IL15, IL6, IL18, IL23, IFN-α, IFN-β, IFN-γ, GM-CSF, M-CSF, RIG-I, MDA5, cGAS, Toll-like receptors, MAVS/VISA, STING/MITA, TRIF, TBK1, IRF3, IRF7, IRF1, JAK1, JAK2, Tyk2, STAT1, STAT2, STAT3, TNFR and any combination thereof.

In a particular embodiment, the added agent is FADD or a mRNA encoding FADD. In a particular embodiment, the added agent is MLKL (or MLKL-4HB) or an mRNA encoding MLKL (or MLKL-4HB). For instance, in one embodiment, the method, use or composition further includes small molecule reagents, recombinant proteins, antibodies.

In some embodiments, the FADD includes the amino acid sequence of SEQ ID NO:15, or a sequencing having at least 70%, 75%, 80%, 85%, 90%, 95%, 97%, 98%, or 99% sequence identity to SEQ ID NO:15. In some embodiment, the mRNA encoding FADD includes SEQ ID NO: 16, or has at least 70%, 75%, 80%, 85%, 90%, 95%, 97%, 98%, or 99% sequence identity to SEQ ID NO:16.

In some embodiments, the MLKL includes the amino acid sequence of SEQ ID NO:17, or a sequencing having at least 70%, 75%, 80%, 85%, 90%, 95%, 97%, 98%, or 99% sequence identity to SEQ ID NO:17 (or residues 1-125 of SEQ ID NO:17). In some embodiment, the mRNA encoding MLKL includes SEQ ID NO:18, or has at least 70%, 75%, 80%, 85%, 90%, 95%, 97%, 98%, or 99% sequence identity to SEQ ID NO:18.

In some embodiments, the added agent is both FADD and MLKL, either encoded in the same mRNA or separate mRNA molecules.

mRNAs may be synthesized according to any of a variety of known methods. For example, the mRNAs may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, SP6 or other RNA polymerase), DNAse I, pyrophosphatase, and/or RNAse inhibitor. The exact conditions will vary according to the specific application.

In some embodiments, for the preparation of mRNA, a DNA template is transcribed in vitro. A suitable DNA template typically has a promoter, for example a T3, T7, SP6 or other RNA polymerase promoter, for in vitro transcription, followed by desired nucleotide sequence for desired mRNA and a termination signal.

Desired mRNA sequence may be determined and incorporated into a DNA template using standard methods. For example, starting from a desired amino acid sequence, a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

The mRNA includes linear RNA, circular RNA and any other form of RNA. The mRNA may be synthesized as unmodified or modified mRNA. In some embodiments, the mRNA is modified to enhance stability. In some embodiments, the mRNA is modified to reduce immunogenicity. In some embodiments, the mRNA is modified to enhance efficiency of translation.

It is contemplated that for certain application of the present technology, the mRNA used are not modified to reduce immunogenicity, which is beneficial to the treatment efficacy. In some embodiments, each mRNA does not include chemical modification that reduces immunogenicity. In some embodiments, each mRNA does not include chemical modification to the backbone. In some embodiments, each mRNA only includes natural nucleosides.

Modifications of mRNA can include, for example, modifications of the nucleotides of the RNA. A modified mRNA can thus include, for example, backbone modifications, sugar modifications or base modifications. In some embodiments, the mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 13-D-mannosyl-queosine, wybutoxosine, and phosphoramidates, phosphorothioates, peptide nucleotides, methylphosphonates, 7-deazaguanosine, 5-methylcytosine and inosine. In some embodiments, at least one of the uridine nucleosides in the mRNAs are chemically modified. In some embodiments, the chemically modified uridine nucleosides are N1-methylpseudouridines.

In some embodiments, the mRNAs may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g., cytidine 5′-O-(1-thiophosphate)), boranophosphates, positively charged guanidinium groups etc., which means by replacing the phosphodiester linkage by other anionic, cationic or neutral groups.

In some embodiments, the mRNAs may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, the mRNAs may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoallyluridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate, N1-methyladenosine 5′-triphosphate, N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

According to various embodiments, the timing of expression of delivered mRNAs can be tuned to suit a particular medical need. In some embodiments, the expression of the OX40L protein encoded by delivered mRNA is detectable 1, 2, 3, 6, 12, 18, 24, 30, 36, 42, 48, 54, 60, 66, and/or 72 hours in serum or target tissues after a single administration of provided liposomes or compositions. In some embodiments, the expression of the OX40L protein encoded by the mRNA is detectable 1 day, 2 days, 3 days, 4 days, 5 days, 6 days, and/or 7 days in serum or target tissues after a single administration of provided liposomes or compositions. In some embodiments, the expression of the OX40L protein encoded by the mRNA is detectable 1 week, 2 weeks, 3 weeks, and/or 4 weeks in serum or target tissues after a single administration of provided liposomes or compositions. In some embodiments, the expression of the protein encoded by the mRNA is detectable after a month or longer after a single administration of provided liposomes or compositions.

In some embodiments, the expression of the IL-12A protein encoded by delivered mRNA is detectable 1, 2, 3, 6, 12, 18, 24, 30, 36, 42, 48, 54, 60, 66, and/or 72 hours in serum or target tissues after a single administration of provided liposomes or compositions. In some embodiments, the expression of the IL12-A protein encoded by the mRNA is detectable 1 day, 2 days, 3 days, 4 days, 5 days, 6 days, and/or 7 days in serum or target tissues after a single administration of provided liposomes or compositions. In some embodiments, the expression of the IL-12A protein encoded by the mRNA is detectable 1 week, 2 weeks, 3 weeks, and/or 4 weeks in serum or target tissues after a single administration of provided liposomes or compositions. In some embodiments, the expression of the protein encoded by the mRNA is detectable after a month or longer after a single administration of provided liposomes or compositions. In some embodiments, the expression of the IL-12B protein encoded by delivered mRNA is detectable 1, 2, 3, 6, 12, 18, 24, 30, 36, 42, 48, 54, 60, 66, and/or 72 hours in serum or target tissues after a single administration of provided liposomes or compositions. In some embodiments, the expression of the IL-12B protein encoded by the mRNA is detectable 1 day, 2 days, 3 days, 4 days, 5 days, 6 days, and/or 7 days in serum or target tissues after a single administration of provided liposomes or compositions. In some embodiments, the expression of the IL-12B protein encoded by the mRNA is detectable 1 week, 2 weeks, 3 weeks, and/or 4 weeks in serum or target tissues after a single administration of provided liposomes or compositions. In some embodiments, the expression of the protein encoded by the mRNA is detectable after a month or longer after a single administration of provided liposomes or compositions.

A specific dosage and treatment regimen for any particular patient will depend upon a variety of factors, including the particular mRNA, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the mRNA used, the severity of the disease, and the desired effect. The amount used can be determined by pharmacological and pharmacokinetic principles well known in the art.

Methods of administration of the mRNA include but are not limited to intradermal, intramuscular, intraperitoneal, intravenous, subcutaneous, intranasal, and epidural. The term “parenteral” as used herein refers to modes of administration which include intravenous, intramuscular, intraperitoneal, intrasternal, subcutaneous and intra-articular injection and infusion. In some embodiments, the administration is intratumoral injection. In some embodiments, the administration is subcutaneous injection. In some embodiments, the administration is intramuscular or intravenous injection. In some embodiments, the administration is subcutaneous injection, intramuscular injection, intraperitoneal injection, thoracic injection, intravenous injection, arterial injection, or a combination thereof.

In some embodiments, the injection is into a tumor tissue. In some embodiments, the injection is into one side, such as an end or a portion, of a tumor tissue. In some embodiments, the injection is not into a tumor tissue.

In some embodiments, the administration is made at a frequency of 3 times a week, twice a weekly, once a week, once every 2 weeks, once every 3 weeks, once every 4 weeks, once a month, or once every 3-6 months.

Cancers that can be suitably treated with the present technology include solid tumors, leukemia and lymphoma. In some embodiments, the cancer is squamous cell carcinoma, lung cancer, peritoneal cancer, hepatocellular carcinoma, gastric cancer, pancreatic cancer, glioblastoma, cervical cancer, ovarian cancer, liver cancer, bladder cancer, urethral cancer, breast cancer, colon cancer, rectal cancer, colorectal cancer, endometrial cancer, uterine cancer, salivary gland cancer, kidney cancer, prostate cancer, vulvar cancer, thyroid cancer, liver cancer, anal cancer, soft tissue sarcoma, neuroblastoma, penile cancer, melanoma, superficial spreading melanoma, lentigines melanoma, acral melanoma, nodular melanoma, multiple bone marrow Tumor, B-cell lymphoma, chronic lymphocytic leukemia, non-Hodgkin's lymphoma, acute lymphoblastic leukemia, hairy cell leukemia, chronic myeloblastic leukemia, post-transplant lymphoproliferative disorders, brain tumors, brain cancer and head and neck cancer.

In one embodiment, the cancer is a solid tumor. In one embodiment, the cancer is metastatic. In one embodiment, the cancer is colon cancer. In one embodiment, the cancer is breast cancer, including triple negative breast cancer. In one embodiment, the cancer is lung cancer.

### Compositions and Combinations

Combinations, packages, kits and compositions are also provided that are useful for carrying out the methods of the instant disclosure.

One embodiment provides a combination, package, kit, or composition that includes a mRNA encoding an OX40 agonist (e.g., OX40L or a protein/polypeptide/antibody or their combination that activates OX40 (e.g., an OX40L protein or anti-OX40 agonist antibody)), and an mRNA encoding IL-12 or a protein/polypeptide/antibody or their combination that acts like IL-12 to stimulate IL-12 receptor (e.g., IL-12 or IL-23 mRNA/protein/polypeptide/antibody). In some embodiments, the mRNA molecule(s) are injected into the subject directly. In some embodiments, one or more or all of the mRNA molecules are delivered as DNA which are then transcribed into mRNA in vivo.

Another embodiment provides a combination, package, kit, or composition that includes a mRNA encoding an OX40 agonist (e.g., OX40L or a protein/polypeptide/antibody or their combination that activates OX40 (e.g., an OX40L protein or anti-OX40 agonist antibody)), and an IL-12 or a protein/polypeptide/antibody or their combination that acts like IL-12 to stimulate IL-12 receptor (e.g., IL-12 or IL-23 mRNA/protein/polypeptide/antibody).

Another embodiment provides a combination, package, kit, or composition that includes an OX40 agonist (e.g., OX40L or a protein/polypeptide/antibody or their combination that activates OX40 (e.g., an OX40L protein or anti-OX40 agonist antibody)), and an mRNA encoding IL-12 or a protein/polypeptide/antibody or their combination that acts like IL-12 to stimulate IL-12 receptor (e.g., IL-12 or IL-23 mRNA/protein/polypeptide/antibody).

Another embodiment provides a combination, package, kit, or composition that includes an OX40 agonist (e.g., OX40L or a protein/polypeptide/antibody or their combination that activates OX40 (e.g., an OX40L protein or anti-OX40 agonist antibody)), and an IL-12 or a protein/polypeptide/antibody or their combination that acts like IL-12 to stimulate IL-12 receptor (e.g., IL-12 or IL-23 protein/polypeptide/antibody).

In any of the above embodiments, combination, package, kit, or composition further includes a FADD protein, an mRNA encoding FADD, or a DNA construct encoding FADD. In any of the above embodiments, combination, package, kit, or composition further includes a MLKL protein, an mRNA encoding MLKL, or a DNA construct encoding MLKL.

In any of the above embodiments, combination, package, kit, or composition further includes a TNFR protein, an mRNA encoding TNFR, or a DNA construct encoding TNFR. In any of the above embodiments, combination, package, kit, or composition further includes a GSDMD protein, an mRNA encoding GSDMD, or a DNA construct encoding GSDMD.

In some embodiments, the OX40L is a human protein. In some embodiments, the OX40L is a full OX40L protein rather than a fragment or domain thereof, such as a soluble portion. In some embodiments, the OX40L is a full OX40L protein with different isoforms rather than a fragment or domain thereof, such as a soluble portion.

In some embodiments, the OX40 agonist is a polypeptide that includes at least an extracellular domain of full-length OX40L, which may be fused to a transmembrane domain and optionally an intracellular fragment of another protein.

In some embodiments, the OX40 agonist is a polypeptide that includes the extracellular domain, either alone or fused with a linker fragment (e.g., oligomerization domain) that can promote the formation of its homo-dimers, homo-trimers or homo-oligomers. Protein domains as such as the Fc fragment of immunoglobulins are commonly used to promote formation of homo-dimers.

Example trimerization domains include the T4 bacteriophage fibritin trimerization motif (T4F), the GCN4 trimeric leucine zipper motif (GCN4), and the human collagen XVIII derived homotrimerization domain (TIE). In some embodiments, the trimerization domain is not longer than 100 amino acids, or not longer than 90, 80, 70, 60, or 50 amino acids.

In some embodiments, the fusion protein further includes a peptide linker between the OX40L extracellular domain and the trimerization domain. In some embodiments, the peptide linker is flexible.

In some embodiments, the distance between an OX40L extracellular domain and the trimerization domain is not longer than 100 amino acids, or not longer than 90, 80, 70, 60, 50, 40, 30, 25, 20, 15 or 10 amino acids. In some embodiments, the peptide linker is from 5 to 50 amino acid residues in length, preferably from 5 to 20 amino acid residues in length.

In some embodiment, the OX40 agonist is an agonist anti-OX40 antibody or an antigen-binding fragment thereof.

In some embodiments, the OX40 agonist mRNA includes the RNA sequence corresponding to the coding sequence of NM_003326 (SEQ ID NO:3). In some embodiments, the OX40L mRNA includes the RNA sequence corresponding to the coding sequence of NM_001297562 (SEQ ID NO:4). In some embodiment, the OX40 agonist mRNA encodes the protein sequence of NP_003317 (SEQ ID NO:1). In some embodiments, the OX40L mRNA encodes the protein sequence of NP_001284491 (SEQ ID NO:2).

In some embodiment, the OX40 agonist mRNA encodes a protein sequence that has at least 70%, 75%, 80%, 85%, 90%, 95%, 98%, or 99% sequence identity to SEQ ID NO:1 or residues 52-183 of SEQ ID NO:1. In some embodiments, the OX40 agonist mRNA encodes a protein sequence that has at least 70%, 75%, 80%, 85%, 90%, 95%, 98%, or 99% sequence identity to SEQ ID NO:2 or residues 2-133 of SEQ ID NO:2. In some embodiments, the protein sequence retains the activity of human OX40L or activates OX40.

In some embodiments, the OX40 agonist mRNA encodes the extracellular domain of NP_003317 (i.e., residues 52-183 of SEQ ID NO:1, or a peptide having at least 70%, 75%, 80%, 85%, 90%, 95%, 98%, or 99% sequence identity to residues 52-183 of SEQ ID NO:1). In some embodiments, the OX40 agonist mRNA encodes the extracellular domain of NP_001284491 (i.e., residues 2-133 of SEQ ID NO:2, or a peptide having at least 70%, 75%, 80%, 85%, 90%, 95%, 98%, or 99% sequence identity to residues 2-133 of SEQ ID NO:2).

In some embodiments, the extracellular domain of OX40L can be fused to the transmembrane domain and optionally an intracellular fragment of another protein, such that the fusion protein can be anchored to the plasma membrane. The transmembrane domain and intracellular fragment can be from any protein, such as a human protein, in particularly those that are expressed on the membranes of cells in a tissue where OX40L is desired to be expressed.

In some embodiments, the IL-12 is human IL-12. In some embodiments, the IL-12 includes IL-12A (p35). In some embodiments, the IL-12 includes IL-12B (p40). In some embodiment, the IL-12 mRNA includes a mRNA encoding IL-12A and a mRNA encoding IL-12B. In some embodiment, the IL-12 mRNA includes a mRNA encoding both IL-12A and IL-12B.

In some embodiments, the IL-12A mRNA includes the mRNA sequence corresponding to the coding sequence of NM_000882 (SEQ ID NO:9). In some embodiments, the IL-12A mRNA encodes the protein sequence of NP_000873 (SEQ ID NO:5), or the mature protein (residues 57-253 of SEQ ID NO:5).

In some embodiments, the IL-12A mRNA includes the mRNA sequence corresponding to the coding sequence of NM_001354582 (SEQ ID NO:10). In some embodiments, the IL-12A mRNA encodes the protein sequence of NP_001341511 (SEQ ID NO: 6), or the mature protein (residues 57-239 of SEQ ID NO:6).

In some embodiments, the IL-12A mRNA includes the mRNA sequence corresponding to the coding sequence of NM_001354583 (SEQ ID NO:11). In some embodiments, the IL-12A mRNA encodes the protein sequence of NP_001341512 (SEQ ID NO: 7), or the mature protein (residues 57-215 of SEQ ID NO:7).

In some embodiments, the IL-12A mRNA includes the mRNA sequence corresponding to the coding sequence of NM_001397992 (SEQ ID NO:12). In some embodiments, the IL-12A mRNA encodes the protein sequence of NP_001384921 (SEQ ID NO: 8), or the mature protein (residues 23-219 of SEQ ID NO:8).

In some embodiments, the IL-12B mRNA includes the mRNA sequence corresponding to the coding sequence of NM_002187 (SEQ ID NO:14). In some embodiments, the IL-12A mRNA encodes the protein sequence of NP_002178 (SEQ ID NO:13), or the mature protein (residues 23-328 of SEQ ID NO:13).

In some embodiments, the OX40L mRNA (or mRNA encoding a protein/polypeptide/antibody or their combination that activates OX40) and the IL-12 mRNA are separate mRNA molecules. In some embodiments, the OX40L (or mRNA encoding a protein/polypeptide/antibody or their combination that activates OX40) coding sequence and the IL-12 coding sequence are included in the same mRNA molecule which can be translated into a fusion protein/polypeptide or separate proteins/polypeptides.

In some embodiments, one of the mRNA encodes OX40L, such as human OX40L. In some embodiments, the encoded protein has the amino acid sequence of SEQ ID NO:1. In some embodiments, the encoded protein has an amino acid sequence that has at least 70%, 80%, 85%, 90%, 95%, 98%, or 99% sequence identity to SEQ ID NO:1. In some embodiments, a fragment of the encoded protein has an amino acid sequence that has at least 90%, 95%, 98%, or 99% sequence identity to a fragment in SEQ ID NO:1. In some embodiments, the encoded protein has the amino acid sequence of SEQ ID NO:2. In some embodiments, the encoded protein has an amino acid sequence that has at least 70%, 80%, 85%, 90%, 95%, 98%, or 99% sequence identity to SEQ ID NO:2. In some embodiments, a fragment of the encoded protein has an amino acid sequence that has at least 90%, 95%, 98%, or 99% sequence identity to a fragment in SEQ ID NO:2. (Someone may make a fusion protein which contains the extracellular domain of OX40L and the intracellular and transmembrane domains from other proteins).

In some embodiments, the mRNA includes the nucleic acid sequence of SEQ ID NO:3. In some embodiments, the mRNA includes the nucleic acid sequence that has at least 70%, 80%, 85%, 90%, 95%, 98%, or 99% sequence identity to SEQ ID NO:3. In some embodiments, the mRNA includes the nucleic acid sequence of SEQ ID NO:4. In some embodiments, the mRNA includes the nucleic acid sequence that has at least 70%, 80%, 85%, 90%, 95%, 98%, or 99% sequence identity to SEQ ID NO:4.

In some embodiments, one of the mRNA encodes IL-12A, such as human IL-12A. In some embodiments, the encoded protein has the amino acid sequence of SEQ ID NO:5. In some embodiments, the encoded protein has an amino acid sequence that has at least 70%, 80%, 85%, 90%, 95%, 98%, or 99% sequence identity to SEQ ID NO:5. In some embodiments, the encoded protein has the amino acid sequence of SEQ ID NO:6. In some embodiments, the encoded protein has an amino acid sequence that has at least 70%, 80%, 85%, 90%, 95%, 98%, or 99% sequence identity to SEQ ID NO:6. In some embodiments, the encoded protein has the amino acid sequence of SEQ ID NO:7. In some embodiments, the encoded protein has an amino acid sequence that has at least 70%, 80%, 85%, 90%, 95%, 98%, or 99% sequence identity to SEQ ID NO:7. In some embodiments, the encoded protein has the amino acid sequence of SEQ ID NO:8. In some embodiments, the encoded protein has an amino acid sequence that has at least 70%, 80%, 85%, 90%, 95%, 98%, or 99% sequence identity to SEQ ID NO: 8.

In some embodiments, the mRNA includes the nucleic acid sequence of SEQ ID NO:9. In some embodiments, the mRNA includes the nucleic acid sequence that has at least 70%, 80%, 85%, 90%, 95%, 98%, or 99% sequence identity to SEQ ID NO:9. In some embodiments, the mRNA includes the nucleic acid sequence of SEQ ID NO:10. In some embodiments, the mRNA includes the nucleic acid sequence that has at least 70%, 80%, 85%, 90%, 95%, 98%, or 99% sequence identity to SEQ ID NO:10. In some embodiments, the mRNA includes the nucleic acid sequence of SEQ ID NO:11. In some embodiments, the mRNA includes the nucleic acid sequence that has at least 70%, 80%, 85%, 90%, 95%, 98%, or 99% sequence identity to SEQ ID NO:11. In some embodiments, the mRNA includes the nucleic acid sequence of SEQ ID NO:12. In some embodiments, the mRNA includes the nucleic acid sequence that has at least 70%, 80%, 85%, 90%, 95%, 98%, or 99% sequence identity to SEQ ID NO: 12.

In some embodiments, one of the mRNA encodes IL-12B, such as human IL-12B. In some embodiments, the encoded protein has the amino acid sequence of SEQ ID NO:13. In some embodiments, the encoded protein has an amino acid sequence that has at least 70%, 80%, 85%, 90%, 95%, 98%, or 99% sequence identity to SEQ ID NO:13.

In some embodiments, the mRNA includes the nucleic acid sequence of SEQ ID NO: 14. In some embodiments, the mRNA includes the nucleic acid sequence that has at least 70%, 80%, 85%, 90%, 95%, 98%, or 99% sequence identity to SEQ ID NO: 14.

In some embodiments, the one or more mRNA encode, collectively OX40L (or a protein/polypeptide/antibody or their combination that activates OX40) and IL-12A. In some embodiments, the one or more mRNA encode, collectively OX40L (or a protein/polypeptide/antibody or their combination that activates OX40) and IL-12B. In some embodiments, the one or more mRNA encode, collectively OX40L (or a protein/polypeptide/antibody or their combination that activates OX40), IL-12A and IL-12B.

When the mRNA molecules encoding IL-12 and OX40L (or a soluble counterpart) are provided on separate molecules, their ratios can be adjusted as needed. In one embodiment, the mass ratio between the IL-12 mRNA and the OX40L mRNA is from 1:0.5 to 1:6, without limitation. In one embodiment, the mass ratio between the IL-12 mRNA and the OX40L mRNA is from 1:0.5 to 1:5. In one embodiment, the mass ratio between the IL-12 mRNA and the OX40L mRNA is from 1:0.5 to 1:4. In one embodiment, the mass ratio between the IL-12 mRNA and the OX40L mRNA is from 1:0.75 to 1:6. In one embodiment, the mass ratio between the IL-12 mRNA and the OX40L mRNA is from 1:0.75 to 1:5. In one embodiment, the mass ratio between the IL-12 mRNA and the OX40L mRNA is from 1:0.75 to 1:4. In one embodiment, the mass ratio between the IL-12 mRNA and the OX40L mRNA is from 1:0.8 to 1:5. In one embodiment, the mass ratio between the IL-12 mRNA and the OX40L mRNA is from 1:0.8 to 1:4. In one embodiment, the mass ratio between the IL-12 mRNA and the OX40L mRNA is from 1:0.8 to 1:3.

In one embodiment, the mass ratio between the IL-12 mRNA and the OX40L mRNA is from 1:0.9 to 1:5. In one embodiment, the mass ratio between the IL-12 mRNA and the OX40L mRNA is from 1:0.9 to 1:4. In one embodiment, the mass ratio between the IL-12 mRNA and the OX40L mRNA is from 1:0.9 to 1:3.5. In one embodiment, the mass ratio between the IL-12 mRNA and the OX40L mRNA is from 1:0.9 to 1:3. In one embodiment, the mass ratio between the IL-12 mRNA and the OX40L mRNA is from 1:0.9 to 1:2.5.

In one embodiment, the mass ratio between the IL-12 mRNA and the OX40L mRNA is from 1:0.9 to 1:2. In one embodiment, the mass ratio between the IL-12 mRNA and the OX40L mRNA is from 1:1 to 1:4. In one embodiment, the mass ratio between the IL-12 mRNA and the OX40L mRNA is from 1:1 to 1:3. In one embodiment, the mass ratio between the IL-12 mRNA and the OX40L mRNA is from 1:1 to 1:2.5. In one embodiment, the mass ratio between the IL-12 mRNA and the OX40L mRNA is from 1:1 to 1:2.

In some embodiments, the OX40L is a full-length OX40L protein. In some embodiments, the OX40L is Fc-OX40L (soluble fragment).

As shown in the experimental examples, addition of other potential therapeutic agents may not increase the therapeutic efficacy of the instant combination of mRNA. Accordingly, in one embodiment, the treatment method or use or composition does not include some other types of mRNA.

In one embodiment, excluded are mRNAs encoding an immune checkpoint inhibitor such as PD-L1, PD-1 and CTLA-4. In one embodiment, excluded are mRNAs encoding an interferon, such as IFN-«, IFN-β, or IFN-γ. In one embodiment, excluded are mRNAs encoding another of the IL-12 family, such as IL-23, IL-27 and IL-35. In one embodiment, excluded are mRNAs encoding other cytokines, such as IL-18.

In some embodiments, each mRNA is a linear mRNA or circular mRNA. In some embodiments, each mRNA further comprises a miRNA binding site. In some embodiments, each mRNA does not include chemical modification that reduces immunogenicity. In some embodiments, the mRNA does not include chemical modification to the backbone. In some embodiments, each mRNA only includes natural nucleosides.

In some embodiments, at least one of the uridine nucleosides in the mRNAs are chemically modified. In some embodiments, the chemically modified uridine nucleosides are N1-methylpseudouridines. In some embodiments, the first mRNA and the second mRNA are formulated with a pharmaceutically acceptable carrier.

### EXAMPLES

### Example 1: Combination of IL-12 and OX40L mRNA Synergistically Inhibited Tumors

This example evaluated the effects of various factors, delivered as synthetic mRNA, in inhibiting the growth of tumors.

The factors included a soluble PD1 (sPD1, as a PD-1/PD-L1 inhibitor), the OX40 ligand (OX40L, isoform 1), IL-12 (a IL12B-IL12A fusion protein), interferon beta (IFN-β), Resiquimod (R-848, a ligand for Toll-like receptor 7/8), and GFP as a control.

A colon cancer mouse model was used in this example. CT-26 is a murine colon cancer cell line. Eight days following implantation of CT-26 cells in mice, an mRNA sample that included individual mRNA or their mixtures (10 μg of each mRNA per mouse) (Table 1), packed in lipid nanoparticles (LNP), was injected into the tumor. At day 18, the animals were sacrificed, tumors were removed, and tumor volumes were measured. The results are shown in FIG. 1.

TABLE 1
mRNA Samples

| No. | mRNA #1 (or R848) | mRNA #2 | mRNA #3 | mRNA #4 |
|---|---|---|---|---|
| 1 | GFP | — | — | — |
| 2 | GFP | GFP | — | — |
| 3 | sPD1 | — | — | — |
| 4 | OX40L | — | — | — |
| 5 | IL-12 | — | — | — |
| 6 | IFN-β | — | — | — |
| 7 | R848 | — | — | — |
| 8 | sPD1 | OX40L | — | — |
| 9 | sPD1 | IL-12 | — | — |
| 10 | OX40L | IL-12 | — | — |
| 11 | IFN-β | OX40L | — | — |
| 12 | IFN-β | IL-12 | — | — |
| 13 | IFN-β | OX40L | IL-12 | — |
| 14 | IFN-β | OX40L | sPD1 | — |
| 15 | IFN-β | sPD1 | IL-12 | OX40L |
| 16 | sPD1 | OX40L | IL-12 | — |
| 17 | R848 | sPD1 | OX40L | — |
| 18 | R848 | OX40L | IL-12 | — |
| 19 | R848 | sPD1 | IL-12 | — |
| 20 | R848 | sPD1 | OX40L | IL-12 |
| 21 | IFN-β | IL-12 | sPD1 | — |
| 22 | GFP | GFP | GFP | — |
| 23 | — | — | — | — |

Interestingly, sPD1 alone exhibited no anti-tumor effect. While OX40L showed some antitumor effects, R848 and IFN-β appeared to be more potent, and IL-12 demonstrated the most potent anti-tumor efficacy as a single agent.

Among all combination treatments, the combination of OX40L and IL-12 (Treatment No. 10) easily stood out as the most efficacious one, with a roughly 10-fold reduction of tumor size. Compared to each of the single agents (Treatment Nos. 4 and 5, respectively), this combination achieved synergistic effects.

Unexpectedly, despite its moderate anti-tumor effect as a single agent, R848 actually decreased other agents' anti-tumor effects when used in combinations. For instance, Treatment No. 18 (R848+OX40L+IL-12) was less efficacious than Treatment No. 10 (OX40L+IL-12), and Treatment No. 20 (R848+sPD1+OX40L+IL-12) was less efficacious than Treatment No. 16 (sPD1+OX40L+IL-12).

### Example 2. Anti-Tumor Effects on Non-Injected Side of Tumor

Based on the results of Example 1, this example further explored the anti-tumor efficacy of the combination mRNA treatment in distal tumor areas.

The animal model used in this example is the same as in Example 1. Ten days following the injections of the LNP-packaged OX40L and IL-12 mRNA (at day 18), the tumor volumes on both sides were measured. As shown in FIG. 2, even though the mRNA was only injected to one side of the tumor block, the anti-tumor effects were substantially similar between both sides.

This example, therefore, demonstrates that the injected mRNA and/or its expressed protein product was able to spread in the tumor tissue, leading to potent inhibition of the entire tumor.

### Example 3. Comparison of Full Length OX40L and Soluble OX40L Fragment

This example compared the efficacy of full length OX40L and its soluble portion in a tumor animal model.

The test agents were produced in the animals by mRNA intratumorally injected. The agents included a control (GFP), IL-12, full-length OX40L protein, and a soluble OX40L fragment fused to an IgG Fc fragment (Fc-OX40L).

CT26 tumor cells (1×10⁶) were implanted to mice, and on days 8 and 11, respectively, test agents were injected to the animals intratumorally (1.5 μg total mRNA per animal). At time of first dosing, the CT26 tumor block was about 4 mm in diameter. The animals were inspected on days 8, 11, 14 and 17 post-implantation. As shown in FIG. 3, overall, the combination of IL-12 and OX40L (full-length or soluble) had higher tumor reduction efficacy than either agent alone, both for local tumors (“Treated”) and at the distal regions (“Distal”). Interestingly, the combination with the soluble OX40L (Fc-OX40L) outperformed the combination with the full-length OX40L.

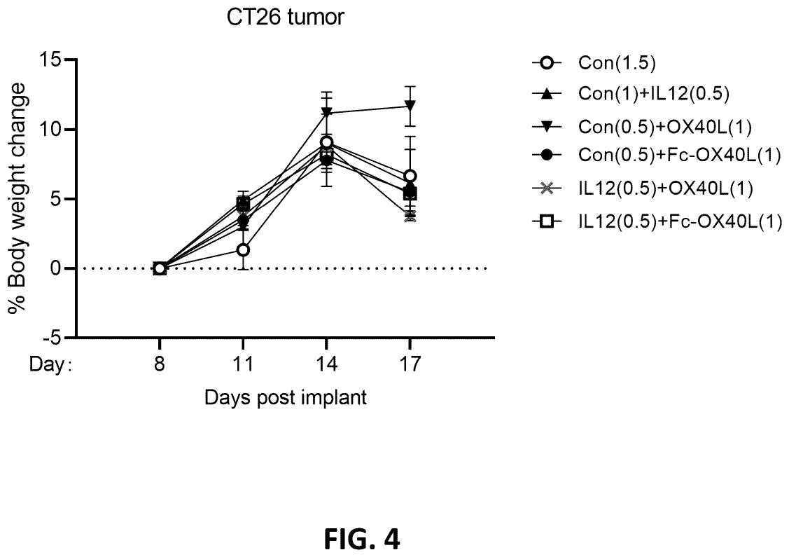

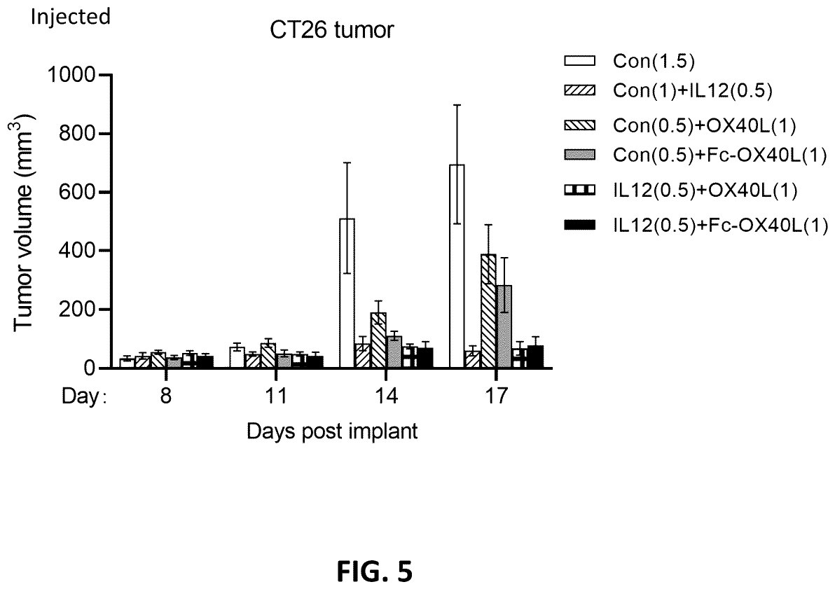

More detailed measurement data of the safety and anti-tumor efficacy of these agents are shown in FIG. 4-6, for days 8, 11, 14 and 17 post-implantation (FIG. 5, injected side; FIG. 6, distal side). All of the test agents exhibited good safety in the animals, comparable to the control (FIG. 4).

These results confirmed that the combination of IL-12 with the soluble Fc-OX40L was the most efficacious.

### Example 4. IL-12, OX12L and MLKL or FADD

This example tested whether adding additional agents could further increase the anti-tumor efficacy of the IL-12/OX40L combination.

Four additional agents were tested, including FADD (FAS-associated death domain protein), MLKL (mixed lineage kinase domain like pseudokinase), GSDMD (Gasdermin D), and TNFR (tumor necrosis factor receptor). FADD is an adaptor protein that bridges members of the tumor necrosis factor receptor superfamily, such as the Fas-receptor, to procaspases 8 and 10 to form the death-inducing signaling complex (DISC) during apoptosis. MLKL plays a role in tumor necrosis factor (TNF)-induced necroptosis via interaction with receptor-interacting protein 3 (RIP3). GSDMD serves as a specific substrate of inflammatory caspases (caspase-1, -4, -5 and -11) and as an effector molecule for the lytic and highly inflammatory form of programmed cell death, pyroptosis.

All of these agents were delivered, in various combinations, to the same animal model as used above, as mRNA (1.5 μg in total), on days 9 and 12, post-tumor cell implantation. While IL-12 and OX40L were encoded on different constructs, the added agent (e.g., FADD) was fused to the OX40L mRNA, through an IRES elements.

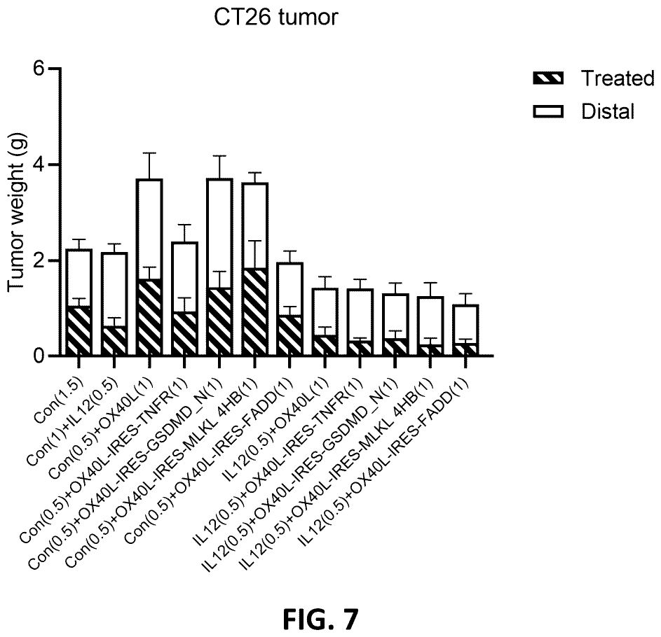

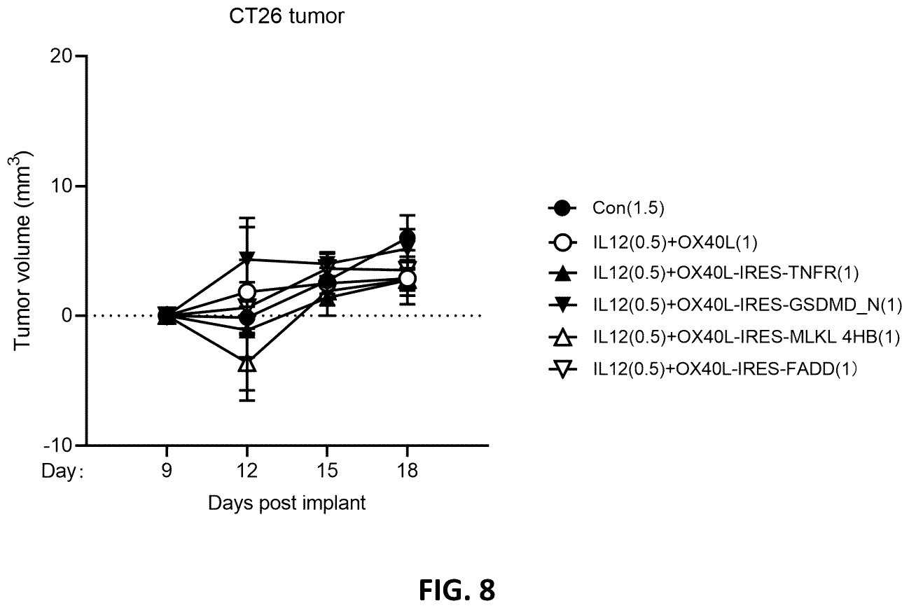

As shown in FIG. 7, the addition of each of MLKL, FADD, GSDMD and TNFR further increased the efficacy of the IL-12/OX12L combination, even though each of them alone did not have observable anti-tumor effects. Among them, the tri-member combinations with the four-helix bundle domain of MLKL or FADD achieved the highest efficacy (FIG. 7, 9). In particular, the IL-12/OX40L/FADD combination exhibited the highest efficacy both at the injection site the distal side of the animals (FIG. 7, 10). All the test agents were safe in the animals (FIG. 8).

### Example 5. Lipid Nanoparticle Formulations

This example tested the anti-tumor effects of IL-12 and OX40L mRNA delivered with various lipid nanoparticle formulations.

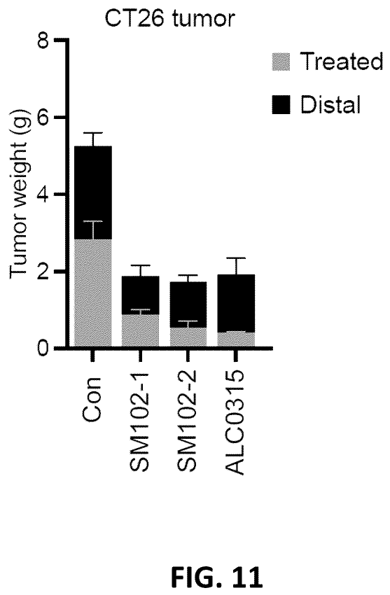

The formulations included formulations SM102-1 and SM102-2 (50% SM102, 38.5% cholesterol, 10% phospholipid, and 1.5% lipid-PEG), and a formulation of ALC0315. Their anti-tumor effects were evaluated with methods as described above. As shown in FIG. 11, all three formulations had comparable, excellent anti-tumor efficacy.

The tissue distribution of these formulations was measured with a mRNA expressing luciferase. Equal amounts luciferase mRNA was intratumorally delivered with SM102-1, SM102-2 and ALC0315. The activity of luciferase was analyzed at 6 hours, 24 hours and 48 hours post-injection.

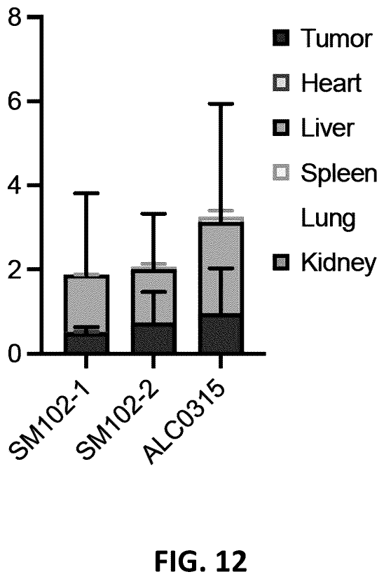

Formulation ALC0315 had lower activity of luciferase at 48 hours post injection but much higher luciferase activity at 6 hours post-injection This suggests that ALC0315-mediated mRNA delivery has shorter duration, but was the most robust, which explains the comparable anti-tumor effects in FIG. 11. For all formulations, most mRNA was detected in the liver while significant portions stayed in the tumor tissues (FIG. 12).

### Example 6. RNA Polymerases, Capping Structures and Polyadenylation Methods

This example tested the efficiency of mRNA prepared by different RNA polymerases, with different cap structures and with different method of polyadenylation.

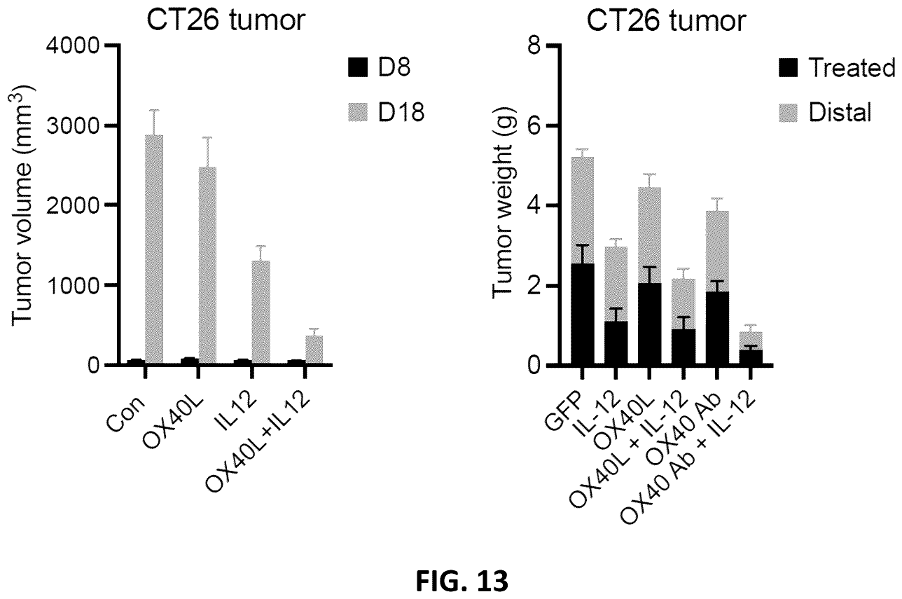

In one sample, the mRNAs of OX40L and IL-12 were transcribed by the VSW-3 RNA polymerase, capped by VCE (vaccinia capping enzyme) and poly(A) catalysed by yeast poly(A) polymerase (post-transcriptional enzyme-based capping and polyadenylation). In a second sample, the mRNAs were transcribed by the T7 RNA polymerase, capped with CleanCap® Reagent AG and polyadenylized with transcription template (co-transcriptional capping and polyadenylation). Both samples were injected to test animals. As shown in FIG. 13, potent anti-tumor effects were observed for the OX40L and IL-12 mRNA produced with different RNA polymerases, capping methods and polyadenylation methods.

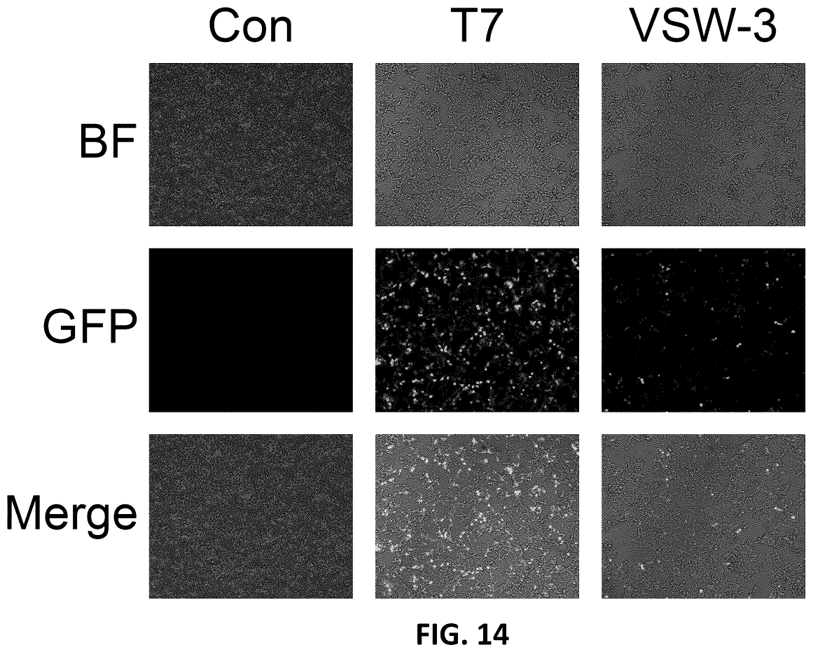

It was reported that RNA products synthesized by the VSW-3 RNA polymerase contained a much lower amount of double-stranded RNA byproducts (dsRNA) than that in T7 RNAP IVT products. This example further tested the compatibility of VSW-3 and CleanCap® Reagent AG. EGFP was used as a tool to indicate the expression of mRNA transcribed by different RNA polymerase. Equal amounts of EGFP mRNAs transcribed by T7 or VSW-3 with CleanCap® Reagent AG were transfected in HEK293T. EGFP mRNAs transcribed by VSW-3 exhibited much lower fluorescence signals than that by T7 (FIG. 14), which indicates VSW-3 has a poor compatibility with CleanCap® Reagent AG using the optimal IVT conditions.

This example then further examined anti-tumor effects of OX40L and IL-12 mRNA with different cap structures. OX40L and IL-12 mRNA with cap-0 or cap-1 was injected at day 8 and day 11 post-tumor implantation. Both cap0 and cap1 mRNA exhibited comparable anti-tumor effects at day 14 but cap1 mRNA has a more constant anti-tumor effects at day 17 than cap0 mRNA (FIG. 15).

### Example 7. mRNA with Different UTR Sequences and Codon Optimization

This example evaluates the effects of different UTR combinations on mRNA translation.

EGFP mRNA was used as a tool for investigation. As shown in FIG. 16, UTR combinations of group H or group R were tested by examining the translation of EGFP mRNA with different UTRs. The group R sequences were adopted from PCT application No. WO2023142283A1, and the 5′UTR and 3′UTR sequences of group H are shown in SEQ ID NO: 19 and 20 (Table A). UTR combinations of group H exhibited a slightly higher MFI (mean fluorescence intensity) than group R.

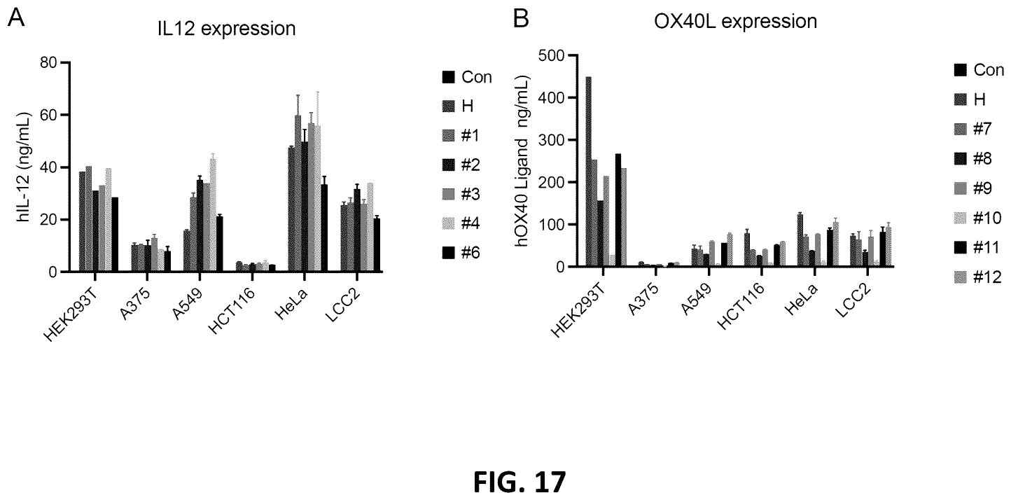

Further, codon optimization was applied to the sequences of OX40L and IL-12 mRNA. As shown in FIG. 17, most of the codon optimized mRNA exhibited excellent translation levels, except #8 (SEQ ID NO:29) and #10 mRNA (SEQ ID NO:31) of OX40L exhibiting a relatively lower translation level.

### Example 8. mRNA with poly(A) of Different Lengths

This example examined the effects of different lengths of poly(A) on EGFP mRNA translation. Equal amounts of EGFP mRNA with 30 adenines or 120 adenines poly(A) tail were transfected into HEK293T. Fluorescence was examined 18 hours post-transfection. EGFP mRNA with 120A had a significant higher translation level than 30A (FIG. 18).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A message RNA (mRNA), comprising a 5′ untranslated region (UTR), a 3′UTR and a coding sequence, wherein the 5′UTR comprises the nucleic acid sequence of SEQ ID NO:19 and the 3′UTR comprises the nucleic acid sequence of SEQ ID NO:20.

2. The mRNA of claim 1, wherein the mRNA comprises a Cap-1 structure at the 5′ end.

3. The mRNA of claim 2, wherein the Cap-1 structure is formed by a CleanCap® co-transcriptional capping reagent.

4. The mRNA of claim 3, wherein the mRNA is transcribed by a T7 RNA polymerase.

5. The mRNA of claim 1, wherein the mRNA further comprises a poly(A) tail, which is preferably prepared by an enzymatic method or a co-transcriptional method.

6. The mRNA of claim 5, wherein the poly(A) comprises at least 50 adenine bases, preferably at least 80 adenine bases, more preferably at least 100 adenine bases.

7. The mRNA of claim 1, wherein the coding sequence encodes IL-12 or an OX40 agonist or both an IL-12 and an OX40 agonist.

8. The mRNA of claim 7, wherein the OX40 agonist is an OX40 ligand (OX40L), a polypeptide comprising the extracellular domain of OX40L, or an agonist anti-OX40 antibody or antigen-binding fragment thereof.

9. The mRNA of claim 7, wherein the coding sequence further encodes FADD (FAS-associated death domain protein), MLKL (mixed lineage kinase domain like pseudokinase) or the four-helix bundle domain of MLKL (MLKL-4HB).

10. The mRNA of claim 7, wherein the coding sequence encoding IL-12 comprises a nucleic acid sequence selected from the group consisting of SEQ ID NO:21-26.

11. The mRNA of claim 7, wherein the coding sequence encoding OX40L comprises a nucleic acid sequence selected from the group consisting of SEQ ID NO:27-33, preferably selected from the group consisting of SEQ ID NO:27, 28, 30, 32 and 33.

12. A pharmaceutical composition comprising the mRNA of claim 1, and an ionizable lipid, a phospholipid, a structural lipid, a polyethylene glycol (PEG) lipid, or a combination thereof.

13. A method for treating cancer in a patient, comprising administering to the patient the mRNA of claim 1.

14. A method for treating cancer in a patient, comprising administering to the patient a first mRNA encoding an OX40 agonist, a second mRNA encoding IL-12, and a third mRNA encoding FADD, MLKL or MLKL-4HB.

15. The method of claim 14, wherein the third mRNA is on the same RNA molecule as the first mRNA or the second mRNA.

16. The method of claim 14, wherein the OX40 agonist is an OX40 ligand (OX40L), a polypeptide comprising the extracellular domain of OX40L, or an agonist anti-OX40 antibody or antigen-binding fragment thereof.

17. The method of claim 14, wherein the first mRNA and the second mRNA are administered at a mass ratio of 4:1 to 0.5:1, preferably 3:1 to 0.75:1, more preferably 2:1 to 0.9:1.

18. The method of claim 13, wherein the administration is subcutaneous injection, intramuscular injection, intraperitoneal injection, thoracic injection, intravenous injection, arterial injection, or a combination thereof.

19. A pharmaceutical composition, comprising a first mRNA encoding an OX40 agonist and/or IL-12, and a second mRNA encoding FADD, MLKL or MLKL-4HB.
